(19) United States
(12) Patent Application Publication
(10) Pub. No.: US 2024/0024216 A1
(43) Pub. Date: Jan. 25, 2024

(54) METHODS AND MATERIALS FOR TREATING HAIR LOSS

(71) Applicant: The Penn State Research Foundation, University Park, PA (US)

(72) Inventors: John Elfar, University Park, PA (US); Jagadeeshaprasad Mashanipalya Guddadarangaiah, University Park, PA (US); Prem Kumar Govindappa, University Park, PA (US)

(21) Appl. No.: 18/270,914

(22) PCT Filed: Jan. 5, 2022
(86) PCT No.: PCT/US2022/011351

§ 371 Date: Jul. 5, 2023

Related U.S. Application Data
(60) Provisional application No. 63/242,824, filed on Sep. 10, 2021.
(60) Provisional application No. 63/134,407, filed on Jan. 6, 2021.

(51) Int. Cl. A61K 8/49; A61K 31/4409; A61Q 7/00

(52) U.S. Cl. CPC A61K 8/4926; A61K 31/4409; A61Q 7/00

(57) ABSTRACT

This document provides methods and materials for treating hair loss. For example, compositions containing 4-aminopyridine (4-AP) and/or one or more derivatives of 4-AP can be administered to a mammal having hair loss to treat the hair loss (e.g., to promote hair growth). Further more, a method for increasing hair regeneration, wherein said method comprises administering a composition comprising 4-AP or one or more derivatives of 4-AP to a mammal identified as being in need of increased hair regeneration.

### CROSS-REFERENCE TO RELATED APPLICATIONS

This application claims the benefit of U.S. Patent Application Ser. No. 63/134,407, filed on Jan. 6, 2021, and U.S. Patent Application Ser. No. 63/242,824, filed on Sep. 10, 2021. The disclosure of the prior applications are considered part of (and are incorporated by reference in) the disclosure of this application.

### BACKGROUND

### 1. Technical Field

This document relates to methods and materials for treating hair loss. For example, compositions containing 4-aminopyridine (4-AP) and/or one or more derivatives of 4-AP can be administered to a mammal having hair loss to treat the mammal.

### 2. Background Information

Hair growth plays a significant role in people's psychosocial lives. Things which negatively affect hair appearance, like hair loss, can be a distressing symptom that can reduce quality of life at any age. In addition to cosmetic issues, loss of hair growth in certain parts of the body can predispose a person to infections.

### SUMMARY

This document provides methods and materials for treating hair loss. For example, compositions containing 4-AP and/or one or more derivatives of 4-AP can be administered to a mammal (e.g., a human) having hair loss to treat the mammal. In some cases, a composition containing 4-AP and/or one or more derivatives of 4-AP can be administered to a mammal (e.g., a human) having hair loss to promote hair growth on the mammal.

As demonstrated herein, administration of 4-AP can increase hair growth and regeneration. Having the ability to stimulate hair regrowth and regeneration as described herein (e.g., by administering a composition containing 4-AP and/or one or more derivatives of 4-AP) provides a unique way to treat hair loss. For example, 4-AP and/or one or more derivatives of 4-AP can be used to treat hair loss associated with one or more hair loss disorders. For example, 4-AP and/or one or more derivatives of 4-AP can be used to treat hair loss associated with one or more medications and/or medical treatments.

In general, one aspect of this document features methods for treating hair loss. The methods can include, or consist essentially of, (a) identifying a mammal as having hair loss, and (b) administering a composition including 4-AP or one or more derivatives of 4-AP to the mammal. The mammal can be a human. The administering can include a systemic administration. The administering can include a local administration. The composition can be effective to deliver about 0.05 mg/kg to about 1 mg/kg of the 4-AP or the one or more derivatives of 4-AP to the mammal. The mammal can have a disease, disorder, or condition associated with the hair loss. The disease, disorder, or condition associated with the hair loss can be traction alopecia, alopecia areata, trichotillomania, a skin graft, or a scar. The mammal can be administered an additional medical treatment. The additional medical treatment can be radiation therapy, a chemotherapy drug, a hormone therapy, Vitamin A, an acne medication, an antibiotic, an antifungal, an anticoagulant, a cholesterol-lowering drug, an immunosuppressant, an anticonvulsant, a blood pressure medication, an antidepressant, or a weight loss drug.

In another aspect, this document features methods for increasing hair growth. The methods can include, or consist essentially of, administering a composition including 4-AP or one or more derivatives of 4-AP to a mammal identified as being in need of increased hair growth. The method can be effective to increase hair growth on the mammal by at least 1.5 fold. The mammal can be a human. The administering can include a systemic administration. The administering can include a local administration. The composition can be effective to deliver about 0.05 mg/kg to about 1 mg/kg of the 4-AP or the one or more derivatives of 4-AP to the mammal. The mammal can have a disease, disorder, or condition associated with the hair loss. The disease, disorder, or condition associated with the hair loss can be traction alopecia, alopecia areata, trichotillomania, a skin graft, or a scar. The mammal can be administered an additional medical treatment. The additional medical treatment can be radiation therapy, a chemotherapy drug, a hormone therapy, Vitamin A, an acne medication, an antibiotic, an antifungal, an anticoagulant, a cholesterol-lowering drug, an immunosuppressant, an anticonvulsant, a blood pressure medication, an antidepressant, or a weight loss drug.

In another aspect, this document features methods for increasing hair regeneration. The methods can include, or consist essentially of, administering a composition including 4-AP or one or more derivatives of 4-AP to a mammal identified as being in need of increased hair regeneration. The method can be effective to increase a number of hair follicles on the mammal by at least 1.5 fold. The mammal can be a human. The administering can include a systemic administration. The administering can include a local administration. The composition can be effective to deliver about 0.05 mg/kg to about 1 mg/kg of the 4-AP or the one or more derivatives of 4-AP to the mammal. The mammal can have a disease, disorder, or condition associated with the hair loss. The disease, disorder, or condition associated with the hair loss can be traction alopecia, alopecia areata, trichotillomania, a skin graft, or a scar. The mammal can be administered an additional medical treatment. The additional medical treatment can be radiation therapy, a chemotherapy drug, a hormone therapy, Vitamin A, an acne medication, an antibiotic, an antifungal, an anticoagulant, a cholesterol-lowering drug, an immunosuppressant, an anticonvulsant, a blood pressure medication, an antidepressant, and a weight loss drug.

In another aspect, this document features methods for promoting proliferation of a cell, wherein said cell is selected from a keratinocyte, a Schwann cell, and fibroblast. The methods can include, or consist essentially of, administering a composition comprising 4-AP or one or more derivatives of 4-AP to a mammal, wherein said keratinocyte, a Schwann cell, or fibroblast of said mammal proliferates. The method can include identifying said mammal as being in need of proliferation of said cell of said mammal prior to said administering. The mammal can be a human. The administering can be a systemic administration. The administering can be a local administration.

In another aspect, this document features methods for promoting migration of a cell, wherein said cell is selected from the group consisting of a keratinocyte, a Schwann cell, and fibroblast. The methods can include, or consist essentially of, administering a composition comprising 4-AP or one or more derivatives of 4-AP to a mammal, wherein said keratinocyte, a Schwann cell, or fibroblast of said mammal migrates. The method can include identifying said mammal as being in need of migration of said cell in the lung of said mammal prior to said administering. The mammal can be a human. The administering can be a systemic administration. The administering can be a local administration.

In another aspect, this document features methods for promoting re-epithelization of a hair follicle in a mammal. The methods can include, or consist essentially of, administering a composition comprising 4-AP or one or more derivatives of 4-AP to a mammal, wherein said hair follicle of said mammal undergoes re-epithelization. The method can include identifying said mammal as being in need of hair follicle re-epithelization prior to said administering. The mammal can be a human. The administering can be a systemic administration. The administering can be a local administration.

In another aspect, this document features methods for promoting hair follicle formation in a mammal. The methods can include, or consist essentially of, administering a composition comprising 4-AP or one or more derivatives of 4-AP to a mammal, wherein a hair-follicle is formed within said mammal. The method can include identifying said mammal as being in need of hair-follicle formation prior to said administering. The mammal can be a human. The administering can be a systemic administration. The administering can be a local administration.

In another aspect, this document features methods for promoting angiogenesis in a hair follicle of a mammal. The methods can include, or consist essentially of, administering a composition comprising 4-AP or one or more derivatives of 4-AP to a mammal, wherein said hair follicle of said mammal undergoes angiogenesis. The method can include identifying said mammal as being in need of hair follicle angiogenesis prior to said administering. The mammal can be a human. The administering can be a systemic administration. The administering can be a local administration.

In another aspect, this document features methods for promoting reinnervation of a hair follicle in a mammal. The methods can include, or consist essentially of, administering a composition comprising 4-AP or one or more derivatives of 4-AP to a mammal, and wherein said hair follicle of said mammal undergoes reinnervation. The method can include identifying said mammal as being in need of hair follicle reinnervation prior to said administering. The mammal can be a human. The administering can be a systemic administration. The administering can be a local administration.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B. Hair growth is promoted by 4-AP. FIG. 1A contains images of mice treated with 4-AP or saline. FIG. 1B contains a graph showing that 4-AP treated mice had increased hair regrowth as compared to saline treated mice.

FIGS. 2A-2B. Hair regeneration is promoted by 4-AP. FIG. 2A contains images of hematoxylin and eosin (H&E) stained skin tissue from mice treated with 4-AP, control (untreated), and saline treated mice. FIG. 2B contains a graph showing that 4-AP treated mice had increased hair regeneration as compared to saline treated mice.

FIGS. 3A-3F. 4-AP accelerates skin wound healing and tissue regeneration. (FIG. 3A) Schematic illustration of the design of animal experiments to test the beneficial therapeutic effect of 4-AP in C57BL/6 mouse wound splinted model. (FIG. 3B) Representative photographs of the wound healing in control (saline treated) and 4-AP treated mice at 0, 3, 5, 7, 9, 12 and 14 days after wounding. Scale bar 1 mm. (FIG. 3C) Percent wound area at each time point relative to the initial wound area in control and 4-AP treated mice. Data show a significant increase in wound closure from day 3 (D3) in 4-AP treated compare to control mice. Graph represent mean±SEM, N=10 wounds/group and *P=0.01 to 0.05, P=0.01 to 0.001, ***P<0.0002, and ****P<0.0001, two-way ANOVA Sidak's multiple comparisons test. (FIG. 3D) Representative images of hematoxylin and eosin (H&E) stained sections of normal control skin and full-thickness excisional wound of saline-control and 4-AP treated skin tissue at day 14 (D14) tissue wounds used for morphometric analysis. Original magnification ×400 μm (FIG. 3E) The thickness of the epidermal determined using H&E stained sections by ImageJ software, measurements reveal thicker epidermis in 4-AP treated compared to saline-control tissue wounds and 4-AP treated mice epidermis was slightly thicker than that of healthy control uninjured skin. (FIG. 3F) The number of hair follicles were counted in H&E-stained images, the mean number of hair follicles were more in 4-AP treated groups compared to saline-control wound tissues and 4-AP treated mice wound tissue had slightly less number of hair follicles than that of healthy control uninjured skin. Mean±SEM, N=5 tissue/group, *P=0.01 to 0.05, P=0.01 to 0.001, ***P<0.0002, and ****P<0.0001, unpaired t-test.

FIGS. 4A-4H. 4-AP enhances fibroblast to myofibroblast formation. (FIG. 4A) Representative histology of Masson's trichrome stained images of healing control and full-thickness excisional wound of saline-control and 4-AP treated wounds on day 14. Original magnification ×200 μm. (FIG. 4B) The Masson's Trichrome stained images used for the collagen density measurement, quantified as average blue pixel density per area in wound healing tissue harvested on day 14. The collagen staining intensity in the 4-AP treated group was significantly higher than that of control. Intensity mean±SEM, N=6 wound tissue/group, *P=0.01 to 0.05, P=0.01 to 0.001, ***P<0.0002, and ****P<0.0001, unpaired t-test. (FIG. 4C) Co-immunofluorescence staining of vimentin (green), α-SMA (red) and nuclear stain (DAPI-blue) in control and 4-AP treated skin wound sections at day 14. Original magnification ×50 μm, intensity mean±SEM, N=6 wound tissue/group, *P=0.01 to 0.05, P=0.01 to 0.001, ***P<0.0002, and ****P<0.0001, unpaired t-test. (FIG. 4D and FIG. 4E) The vimentin and α-SMA stained images were assessed for quantitative density measurement, 4-AP treated group showed higher intensity for both stains compare to saline control mice group. (FIG. 4F) A representative western blots of α-SMA and TGF-β levels, 4-AP treatment induces more myofibroblast differentiation-α-SMA expression due to TGF-β in the wound healing tissue. Quantitation of α-SMA and TGF-β levels to normalized to GAPDH (fold change mean±SEM; N=3 wound tissue/group, unpaired t-test. (FIG. 4G) Immunofluorescent TGF-β stained wound tissue sections. 4-AP treatment induces TGF-β (green) detected by immunostaining, nuclei stained with DAPI (blue) with scale bar=50 μm. (FIG. 4H) Quantitative integrated density analysis of TGF-β. Fold change mean±SEM;N=5 wound mice/group, *P=0.01 to 0.05, P=0.01 to 0.001, ***P<0.0002, and ****P<0.0001, unpaired t-test.

FIGS. 5A-5I. 4-AP promotes cutaneous wound healing and tissue regeneration associated with keratinocytes proliferation and migration epithelial stem cell markers during wound healing. (FIG. 5A) Keratinocytes proliferation measured by immunostaining of K14, 4-AP treated mice exhibited more intense K14 (green) stain in epidermis and neo-hair follicles compared to control wound mice, nuclear stain DAPI (blue). (FIG. 5B and FIG. 5C) Percent and area of K14 positive cells in control and 4-AP treated skin wounds at day 14; original magnification ×50 μm, mean±SEM; N=4 tissue wounds/group, *P=0.01 to 0.05, P=0.01 to 0.001, ***P<0.0002, and ****P<0.0001, unpaired t-test. (FIG. 5D) Representative keratinocyte hyper-proliferative marker (K17) IHC images of control and 4-AP treated skin wounds. (FIG. 5E and FIG. 5F) Percent and area of K17 positive cells in control and 4-AP treated skin wounds at day 14; K17 expressing cells were more abundant in the 4-AP treated group than that in the control group. Original magnification ×50 μm, mean±SEM; N=6 tissue wounds/group, *P=0.01 to 0.05, P=0.01 to 0.001, ***P<0.0002, and ****P<0.0001, unpaired t-test. (FIG. 5G) Keratinocytes epithelial-stem cells measured by immunostaining of K15, 4-AP treated mice showed higher intense K15 (green) stain compared to control wound mice, nuclear stain DAPI (blue). (FIG. 5H and FIG. 5I) Percent and area of K14 positive cells in control and 4-AP treated skin wounds at day 14; original magnification ×100 μm, Mean±SEM; N=4 tissue wounds/group, *P=0.01 to 0.05, P=0.01 to 0.001, ***P<0.0002, and ****P<0.0001, unpaired t-test.

FIGS. 6A-6E. 4-AP positively effects stem-cell proliferation through reinnervation and enhanced neuropeptide elaboration. (FIG. 6A) A representative Co-immunostaining analysis of Ki-67+ proliferating keratinocytes and NF-H of innervated hair follicles seen in the wound at day 14; original magnification ×20 μm. (FIG. 6B and FIG. 6C) 4-AP treated animal wounds Ki67⁺ cells were significantly increased percentage of Ki-67+ proliferating cells and area of NF-H in the 4-AP treated wound tissue compared to the control. Ki67+ cells percent and area was calculated and expressed in mean±SE; N=4 tissue wounds/group, *P=0.01 to 0.05, P=0.01 to 0.001, ***P<0.0002, and ****P<0.0001, unpaired t-test, while NF-H expression area was measured and represented in mean±SEM; N=4 tissue wounds/group, *P=0.01 to 0.05, P=0.01 to 0.001, ***P<0.0002, and ****P<0.0001, unpaired t-test. (FIG. 6D) Immunofluorescence staining of wound skin tissue sections for pan-neuronal marker PGP-9.5 (red) and nuclear stain DAPI. (FIG. 6E) Quantitative intensity analysis of PGP-9.5 protein expressing cells in wound healing tissue, the analysis shows significantly increased PGP-9.5 intensity in 4-AP treated group than that of control group mice at day 14 and data represented as percent mean±SEM; N=6 tissue wounds/group, *P=0.01 to 0.05, P=0.01 to 0.001, ***P<0.0002, and ****P<0.0001, unpaired t-test.

FIGS. 7A-7F. 4-AP enhances the Schwann cells proliferation and de-differentiation and promotes reinnervation. (FIG. 7A) Co-immunostaining of S100 (green) p75-NTR (red) of wound sections shows increased expression of the Schwann cells and de-differentiation marker at day 14; original magnification ×50 μm. (FIG. 7B and FIG. 7C) Quantitative analysis of S100+ and p75-NTR expressing cells in percentage of wound healing tissue shows increase of both S100 and p75+ cells in 4-AP treated group than that of control group mice at day 14 and data represented as percent mean±SEM; N=6 tissue wounds/group, *P=0.01 to 0.05, P=0.01 to 0.001, ***P<0.0002, and ****P<0.0001, unpaired t-test. (FIG. 7D) A representative western blot of p75-NTR and GAPDH. (FIG. 7E and FIG. 7F) Normalized integrated densities for p75-NTR levels mean±SEM; N=2 wound tissue/group, *p<0.05, unpaired t-test.

FIGS. 8A-8G. 4-AP promotes transcription factor, neurotrophic factor, and neuro peptides due to reinnervation. (FIG. 8A) Immunofluorescence triple co-staining of wound skins for the transcription factor SOX10 (green), neuropeptide substance-P (yellow), neutotrophic factor NGF (red), and nuclear stain DAPI and original magnification ×50 μm. (FIG. 8B, FIG. 8C, and FIG. 8D) Quantitative analysis of SOX10, substance-P and NGF expressing cells in percentage of wound healing tissue, the analysis showed significantly increased intensities of SOX10, substance-P and NGF expressing cells in 4-AP treated group than that of control group mice on day 14 and data represented as percent mean±SEM; N=6 tissue wounds/group, *P=0.01 to 0.05, P=0.01 to 0.001, ***P<0.0002, and ****P<0.0001, unpaired t-test. (FIG. 8E) A representative western blot of SOX10, NGF and GAPDH. (FIG. 8F and FIG. 8G) Normalized integrated densities for SOX10 and NGF levels mean±SEM; N=3 wound tissue/group, *P=0.01 to 0.05, P=0.01 to 0.001, ***P<0.0002, and ****P<0.0001, unpaired t-test.

FIGS. 9A-9F. Effect of 4-AP exposure on primary keratinocyte scratch wound healing assay. (FIG. 9A) The scratch wounds were generated on confluent keratinocytes. Micrographs show representative images of no treatment and 4-AP exposed cells in-vitro. The keratinocytes migration and wound closure were assessed for every hour until 42 hours post-scratching. The yellow lines indicate the wound borders at the beginning of the assay and were recorded every hour until 42 hours. Selected hours such as 0, 3, 6, 9, 12, 18, 24 and 42 hours are represented. Scale bar, 100 μM. (FIG. 9B) The relative percentage of wound closure was calculated as the ratio of the remaining wound gap at the given time point to initial wound gap at 0 hour. The area of migration and wound closure were measured in five replicates wells using human foreskin primary keratinocytes and the percentage of wound closure data expressed as means±SEM, N=5 wound scratch replicates/group, and *P=0.01 to 0.05, P=0.01 to 0.001, ***P<0.0002, and ****P<0.0001 one-way ANOVA Sidak's multiple comparisons test. (FIG. 9C-FIG. 9F) A representative western blot and normalized integrated densities for SOX10, NGF and GAPDH.

FIGS. 10A-10F. Effect of 4-AP exposure on co-cultured primary keratinocytes either with dermal Schwann cells or dermal fibroblasts scratch wound healing assay. (FIG. 10A) The scratch wounds were generated on confluent primary keratinocytes and dermal Schwann cells. Micrographs show representative images of no treatment and 4-AP exposed cells in-vitro. Co-culture cells migration and wound closure were assessed for every hour until 24 hours post-scratching. The yellow lines indicate the wound borders at the beginning of the assay and were recorded every hour until 24 hours. Selected hours such as 0, 3, 6, 9, 12, 18, and 24 hours are represented. Scale bar, 100 μM. (FIG. 10B) The relative percentage of cells migration and wound closure were measured in four replicates wells and the percentage of wound closure data expressed as means±SEM, N=4 wound scratch replicates/group, and *P=0.01 to 0.05, P=0.01 to 0.001, ***P<0.0002, and ****P<0.0001 one-way ANOVA Sidak's multiple comparisons test. (FIG. 10C) The scratch wounds were generated on confluent primary keratinocytes and dermal fibroblasts. Micrographs show representative images of no treatment and 4-AP exposed cells in-vitro. Co-culture cells migration and wound closure were assessed for every hour until 24 hours post-scratching. The yellow lines indicate the wound borders at the beginning of the assay and were recorded every hour until 24 hours. Selected hours such as 0, 3, 6, 9, 12, 18, and 24 hours are represented. Scale bar, 100 μM. (FIG. 10D) The relative percentage of cells migration and wound closure were measured in four replicates wells and the percentage of wound closure data expressed as means±SEM, N=4 wound scratch replicates/group, and *P=0.01 to 0.05, P=0.01 to 0.001, ***P<0.0002, and ****P<0.0001 one-way ANOVA Sidak's multiple comparisons test. (FIG. 10E) Micrographs show representative images of 4-AP, NGF and combination of 4-AP & NGF with and without NGF antibody exposed cells in-vitro. The keratinocytes migration and wound closure were assessed for every hour until 24 hours post-scratching. The yellow lines indicate the wound borders at the beginning of the assay and were recorded every hour until 24 hours. Selected hours such as 0, 3, 6, 9, 12, 18, and 24 hours are represented. Scale bar, 100 μM. (FIG. 10F) The relative percentage of wound closure was calculated as the ratio of the remaining wound gap at the given time point to initial wound gap at 0 hour. The area of migration and wound closure were measured in four replicates wells using human foreskin primary keratinocytes and the percentage of wound closure data expressed as means±SEM, N=4 wound scratch replicates/group, and *P=0.01 to 0.05, P=0.01 to 0.001, ***P<0.0002, and ****P<0.0001 one-way ANOVA Sidak's multiple comparisons test.

FIGS. 11A-11C. 4-AP decreases inflammatory responses of wound healing. (FIG. 11A) Immunohistochemistry was performed to identify inflammatory (IL-1β) and macrophages (F4/80) in wounds, more inflammatory cells and macrophages are present in granulation tissue of control wounds; in 4-AP treated granulated tissue showed less of inflammatory cells and macrophages at day 14. Original magnification ×100 μm. (FIGS. 11B and 11C) Morphometric quantification of IL1-β and F4/80. The data are shown as the intensity mean±SEM, N=6 wound mice/group and *P=0.01 to 0.05, P=0.01 to 0.001, ***P<0.0002, and ****P<0.0001 unpaired t-test.

FIGS. 12A-12F. 4-AP induces neo-angiogenesis and neuronal peptide wound healing and did not alter keratinocyte K10 expression. (FIG. 12A) Keratin 10 protein expression in healed epidermis by immunofluorescence. 4-AP treatment did not cause any change in expression of keratinocyte K10 expression. Scale bars=50 μm. (FIG. 12B) Percent of K10+ cells in control and 4-AP treated skin wounds at day 14. Mean±SEM; N=4 animals wound tissue/group and *P=0.01 to 0.05, **P=0.01 to 0.001, ***P<0.0002, and ****P<0.0001 unpaired t-test. (FIG. 12C) Immunofluorescence staining of control and healed wound sections for pan-neuronal marker PGP-9.5 (red) and nuclear stain DAPI (blue). Scale bars=20 μm. (FIG. 12D) Quantification of PGP-9.5 protein expressing cells showed significantly increased PGP-9.5 intensity in the 4-AP treated group compared to the saline treated group at day 14. PGP 9.5 in 4-AP-treated mice was not significantly different from seen in uninjured (control) tissue. Mean±SEM; N=6 animal wound tissues/group and 4 control tissue, *P=0.01 to 0.05, **P=0.01 to 0.001, ***P<0.0002, and ****P<0.0001, unpaired t-test. (FIG. 12E) The cutaneous wounded sections were stained with CD31 antibody, and (FIG. 12F) quantitative integrated density analysis of CD31 level was performed. Original magnification ×100 μm. The data are shown as the intensity mean SEM, N=6 wound mice/group and *P=0.01 to 0.05, P=0.01 to 0.001, ***P<0.0002, and ****P<0.0001 unpaired t-test.

FIGS. 13A-13F. Isolation and characterization of keratinocytes, Schwann cells, and fibroblasts from the human neonatal foreskin. The foreskin sample was cut into small pieces and incubated with dispase I. The epidermis and dermis layer was separated. The separated epidermis layer was incubated with trypsin for the isolation of keratinocytes. Isolated keratinocytes were cultured and after reaching 80% confluence subculture for the characterization using specific antibodies by immunofluorescence stain. Using dermis layer was incubated with collagenase and culture the isolated cells in either Schwann cells (DMEM) and/or fibroblast medium. After plating of the Schwann cells for 24 hours, Schwann cells were treated with cytosine arabinose for one day, and the Schwann cells were continued culture in Schwann cell media along with Schwann cells growth factors. (FIG. 13A) Keratinocytes were characterized using keratin 14 (K14, keratinocytes proliferative marker) and K10 (keratin 10, differentiation marker). (FIG. 13B) Schwann cells were characterized using S100 (Schwann cells marker), p75-NTR (nerve growth factor receptor marker) and MPZ (myelin basic protein, myelination marker). (FIG. 13C) Fibroblasts were characterized using vimentin (fibroblast marker) and α-smooth muscle actin (fibroblast differentiation, myofibroblast marker). Original magnification ×100 m for keratinocytes and ×50 μm for both Schwann cells and keratinocytes. (FIG. 13D-FIG. 13F) Cell viability using MTT assay with different concentrations of 4-AP (ranging from 1 to 10000 μM) for (FIG. 13D) Keratinocytes, (FIG. 13F) Schwann cells and (FIG. 13F) fibroblast. The results were expressed as mean±SEM, N=3 replicates/concentration.

FIGS. 14A-14C) The percentage of migration of keratinocytes. (FIG. 14A) Human foreskin isolated keratinocytes used to perform wound healing assay in presence and absence of 4-AP. The relative percent of migration of keratinocytes towards the scratch wound measured every 1 hours until 42 hours using incucyte instrument. The migration was quantified by measuring the distance between the edges and the closest keratinocytes of the wound. The data are shown as the percentage mean±SEM, N=5 wound scratch replicates/group. (FIG. 14B) Human foreskin dermis isolated Schwann cells used to perform wound healing assay in presence and absence of 4-AP. The relative percent of migration of Schwann cells towards the scratch wound measured every 1 hours until 24 hours using incucyte instrument. The migration was quantified by measuring the distance between the edges and the closest Schwann cells of the wound. The data are shown as the percentage mean±SEM, N=4 wound scratch replicates/group. (FIG. 14C) Human foreskin dermis isolated fibroblasts used to perform wound healing assay in presence and absence of 4-AP. The relative percent of migration of fibroblasts towards the scratch wound measured every 1 hours until 24 hours using incucyte instrument. The migration was quantified by measuring the distance between the edges and the closest fibroblasts of the wound. The data are shown as the percentage mean±SEM, N=4 wound scratch replicates/group.

FIG. 15. Co-immunostained of primary keratinocytes exposed to 4-AP and no treatment for 24 hours and stained with keratin 14 (proliferation-K14-green), keratin 10 (differentiation-K10-red) and keratin 15 (hyperproliferation-K17-yellow). DAPI (blue) was used as nuclear counterstaining.

FIGS. 16A-16. Effect of 4-AP exposure on primary dermal fibroblasts scratch wound healing assay. (FIG. 16A) The scratch wounds were generated on confluent fibroblasts. Micrographs show representative images of no treatment and 4-AP exposed cells in vitro. The fibroblast migration and wound closure were assessed for every hour until 24 hours post-scratching. The yellow lines indicate the wound borders at the beginning of the assay and were recorded every hour until 24 hours. Selected hours such as 0, 3, 6, 9, 12, 18, and 24 hours are represented. Scale bar, 100 μM. (FIG. 16B) The relative percentage of cells migration and wound closure were measured in four replicates wells using human foreskin dermal primary fibroblast and the percentage of wound closure data expressed as means±SEM, N=4 wound scratch replicates/group. The fibroblasts did not show any significant difference between 4-AP versus no treatment. (FIG. 16C) Co-immunostained dermal fibroblast exposed to 4-AP and no treatment for 72 hours and stained with fibroblast marker (vimentin-red), myofibroblast differentiation marker (α-SMA-green). DAPI (blue) was used as nuclear counterstaining.

FIGS. 17A-17F. Effect of 4-AP exposure on primary dermal Schwann cells scratch wound healing assay. (FIG. 17A) The scratch wounds were generated on confluent dermal Schwann cells. Micrographs show representative images of no treatment and 4-AP exposed cells in vitro. The Schwann cells migration and wound closure were assessed for every hour until 24 hours post-scratching. The yellow lines indicate the wound borders at the beginning of the assay and were recorded every hour until 24 hours. Selected hours such as 0, 3, 6, 9, 12, 18, and 24 hours are represented. Scale bar, 100 μM. (FIG. 17B) The relative percentage of cells migration and wound closure were measured in four replicates wells using human foreskin dermal primary Schwann cells and the percentage of wound closure data expressed as means±SEM, N=4 wound scratch replicates/group, and *P=0.01 to 0.05, P=0.01 to 0.001, ***P<0.0002, and ****P<0.0001 one-way ANOVA Sidak's multiple comparisons test. (FIG. 17C) Co-immunostained of dermal Schwann cells exposed to 4-AP and no treatment for 72 hours and stained with Schwann cells marker (S100-green), Schwann cells de-differentiation marker (p75-NTR-red) and myelin basic protein (MBP-yellow). DAPI (blue) was used as nuclear counterstaining. (FIG. 17D-FIG. 17F) A representative western blot and normalized integrated densities for SOX10, p75-NTR, NGF and GAPDH.

FIGS. 18A-18C The percentage of migration of keratinocytes in co-culture setting either with Schwann cells and fibroblasts. Human foreskin epidermis isolated keratinocytes were co-cultured with dermis isolated Schwann cells (10:1 ratio; FIG. 18A) and with dermis isolated fibroblasts (10:1 ratio; FIG. 18B). The co-cultured cells were used to perform wound healing assay in presence and absence of 4-AP. The relative percent of migration of cells towards the scratch wound measured every 1 hours until 24 hours using incucyte instrument. The migration was quantified by measuring the distance between the edges and the closest cells of the wound. The data are shown as the percentage mean±SEM, N=4 wound scratch replicates/group. (FIG. 18C) Effect of 4-AP, nerve growth factor (NGF), and combination of 4-AP & NGF in presence and absence of anti-NGF antibody exposure on scratch wound assay performed using foreskin isolated keratinocyte. The relative percent of migration of cells towards the scratch wound measured every 1 hours until 24 hours using incucyte instrument. The migration was quantified by measuring the distance between the edges and the closest keratinocytes of the wound. The data are shown as the percentage mean±SEM, N=4 wound scratch replicates/group.

### DETAILED DESCRIPTION

This document provides methods and materials for treating hair loss. For example, compositions containing 4-AP and/or one or more derivatives of 4-AP can be administered to a mammal (e.g., a human) having hair loss to treat the mammal. In some cases, a composition containing 4-AP and/or one or more derivatives of 4-AP can be administered to a mammal (e.g., a human) having hair loss to promote hair growth on the mammal.

In some cases, a composition described herein (e.g., a composition containing 4-AP and/or one or more derivatives of 4-AP) can be administered to a mammal (e.g., a human) in need thereof (e.g., a human having hair loss) to promote hair growth on the mammal. For example, a composition described herein (e.g., a composition containing 4-AP and/or one or more derivatives of 4-AP) can be administered to a mammal having hair loss to increase hair growth on the mammal by, for example, 10, 20, 30, 40, 50, 60, 70, 80, 90, 95, or more percent. For example, a composition described herein (e.g., a composition containing 4-AP and/or one or more derivatives of 4-AP) can be administered to a mammal having hair loss to increase hair growth on the mammal by, for example, at least 1.5 fold (e.g., about 1.5 fold, about 2 fold, about 2.5 fold, about 3 fold, about 3.5 fold, about 4 fold, about 5 fold, about 6 fold, or more).

In some cases, a composition described herein (e.g., a composition containing 4-AP and/or one or more derivatives of 4-AP) can be administered to a mammal (e.g., a human) in need thereof (e.g., a human having hair loss) to promote hair regeneration on the mammal. For example, a composition described herein (e.g., a composition containing 4-AP and/or one or more derivatives of 4-AP) can be administered to a mammal having hair loss to increase the number of hair follicles on the mammal by, for example, 10, 20, 30, 40, 50, 60, 70, 80, 90, 95, or more percent. For example, a composition described herein (e.g., a composition containing 4-AP and/or one or more derivatives of 4-AP) can be administered to a mammal having hair loss to increase the number of hair follicles on the mammal by, for example, at least 1.5 fold (e.g., about 1.5 fold, about 2 fold, about 2.5 fold, about 3 fold, about 3.5 fold, about 4 fold, about 5 fold, about 6 fold, or more).

Any appropriate mammal having hair loss can be treated as described herein (e.g., by administering a composition containing 4-AP and/or one or more derivatives of 4-AP). Examples of mammals that can have hair loss and that can be treated as described herein include, without limitation, humans, non-human primates such as monkeys, horses, bovine species, porcine species, dogs, cats, mice, rats, rabbits, and goats. In some cases, a mammal having hair loss can be immunocompromised. In some cases, a human having hair loss can be treated as described herein.

A mammal having any type of hair loss can be treated as described herein (e.g., by administering a composition containing 4-AP and/or one or more derivatives of 4-AP). Hair loss can be permanent hair loss or temporary hair loss. Hair loss can be sudden hair loss or gradual hair loss. In some cases, hair loss can be naturally occurring. Examples of types of naturally occurring hair loss that can be treated as described herein include, without limitation, hereditary hair loss (e.g., androgenic alopecia, male-pattern baldness, and female-pattern baldness), hair loss associated with hormonal changes (e.g., hormonal changes due to pregnancy, childbirth, menopause and thyroid problems), stress related hair loss, and hair loss associated with shaving (e.g., hair loss associated with folliculitis caused by shaving). In some cases, hair loss can be associated with one or more diseases, disorders, and/or conditions. Examples of diseases, disorders, and conditions that can be associated with hair loss that can be treated as described herein include, without limitation, traction alopecia (e.g., hair loss associated with hairstyles that pull hair tight, such as pigtails or cornrows), alopecia areata, trichotillomania, the presence of one or more skin grafts (e.g., one or more surgical skin grafts), and scars (e.g., keloid scars). In some cases, hair loss can be associated with (e.g., can be a side effect of and/or adverse effect of) a medication and/or medical treatment. Examples of medications and medical treatments that can be associated with hair loss that can be treated as described herein include, without limitation, radiation therapy (e.g., radiation therapy to the head), chemotherapy drugs (e.g., carboplatin, cisplatin, docetaxel, doxorubicin, and fluorouracil (5-FU)), hormone therapies (e.g., hormone replacements), Vitamin A, acne medications (e.g., vitamin A-derived acne medications such as isotretinoin (e.g., ACCUTANE®) and tretinoin (e.g., RETIN-A®)), antibiotics, antifungals (e.g., voriconazole), anticoagulants (e.g., heparin and warfarin), cholesterol-lowering drugs (e.g., simvastatin and atorvastatin), immunosuppressants (e.g., methotrexate, leflunomide, cyclophosphamide, and etanercept), anticonvulsants (e.g., valproic acid and trimethadione), blood pressure medications (e.g., beta blockers such as metoprolol, timolol, propranolol, atenolol, and nadolol), ACE inhibitors (e.g., enalapril, lisinopril, and captopril), antidepressants (e.g., paroxetine hydrochloride, sertraline, protriptyline, amitriptyline, and fluoxetine), and weight loss drugs.

Hair loss that can be treated as described herein (e.g., by administering a composition containing 4-AP and/or one or more derivatives of 4-AP) can appear in any manner. For example, hair loss can appear as thinning hair (e.g., thinning hair on the top of the head of a mammal such as a human), receding hair (e.g., receding hair at the hairline on the forehead of a mammal such as a human), a broadening of the part in the hair, and/or patchy (e.g., circular) bald spots.

Hair loss that can be treated as described herein (e.g., by administering a composition containing 4-AP and/or one or more derivatives of 4-AP) can affect any location on a mammal. In some cases, hair loss that can be treated as described herein can be on the scalp (e.g., the top of the head) of a mammal. In some cases, hair loss that can be treated as described herein can be on the face of a mammal. In some cases, hair loss that can be treated as described herein can be on the whole body of a mammal. In some cases, hair loss that can be treated as described herein can be on grafted skin present on a mammal.

In some cases, methods described herein also can include identifying a mammal as having hair loss. Examples of methods for identifying a mammal as having hair loss include, without limitation, physical examinations (e.g., pull tests to see how many hairs come out), blood tests (e.g., to identify medical conditions that can cause hair loss), microscopy (e.g., to examine the hair shafts, hair roots, pigment intensity, and/or hair regrowth length), follicle counts, and biopsy follicle assessments. Once identified as having hair loss, a mammal can be administered or instructed to self-administer a composition described herein (e.g., a composition containing 4-AP and/or one or more derivatives of 4-AP).

A composition described herein (e.g., a composition containing 4-AP and/or one or more derivatives of 4-AP) can include 4-AP and/or any appropriate derivative(s) of 4-AP. Examples of derivatives of 4-AP that can be included in a composition described herein include, without limitation, 3,4-diaminopyridine, 3-hydroxy-4-aminopyridine, N-(4-pyridyl)-t-butyl carbamate, N-(4-pyridyl) ethyl carbamate, N-(4-pyridyl) methyl carbamate, and N-(4-pyridyl) isopropyl carbamate. In some cases, 4-AP and/or one or more derivatives of 4-AP can have a structure according to Formula I.

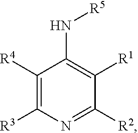

where R¹, R², R³, R⁴, and R⁵ are each independently selected from hydrogen, halogen, amine, hydroxyl, alkoxy, carboxyl, or C1-C6 alkyl. For example, R¹, R², R³, R⁴, and R⁵ can all be hydrogen. In some cases, 4-AP or a derivative thereof can be a potassium channel blocker. In some cases, 4-AP or a derivative thereof can be a calcium channel agonist. In some cases, 4-AP or a derivative thereof can be electrically active. In some cases, 4-AP or a derivative thereof can be in the form of a free base. In some cases, 4-AP or a derivative thereof can be in the form of a salt (e.g., pharmaceutically acceptable salt). When 4-AP or a derivative thereof is in the form of a salt, the salt can include any appropriate acid (e.g., an organic acid or an inorganic acid). Examples of acids that can be used to form a salt with 4-AP or a derivative thereof include, without limitation, hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, and mandelic acid.

In some cases, 4-AP and/or one or more derivatives of 4-AP can be as described elsewhere (see, e.g., U.S. Patent Application Publication No. 2018/0271847 and U.S. Pat. No. 9,993,429).

A composition described herein (e.g., a composition containing 4-AP and/or one or more derivatives of 4-AP) can include any appropriate amount of 4-AP and/or one or more derivatives of 4-AP. In some cases, a composition described herein can include from about 0.5 μM to about 10 μM (e.g., from about 0.5 μM to about 8 μM, from about 0.5 μM to about 6 μM, from about 0.5 μM to about 5 μM, from about 0.5 μM to about 3 μM, from about 0.5 μM to about 2 μM, from about 0.5 μM to about 1 μM, from about 1 μM to about 10 PM, from about 2 μM to about 10 μM, from about 4 μM to about 10 μM, from about 5 μM to about 10 μM, from about 7 μM to about 10 μM, from about 9 μM to about 10 μM, from about 1 μM to about 9 μM, from about 2 μM to about 8 μM, from about 3 μM to about 7 μM, from about 4 μM to about 6 μM, from about 1 μM to about 3 μM, from about 2 μM to about 4 μM, from about 3 μM to about 5 μM, from about 5 μM to about 7 μM, from about 6 μM to about 8 μM, or from about 7 μM to about 9 μM) of 4-AP and/or one or more derivatives of 4-AP. In some cases, a composition described herein can include from about 0.01% to about 99% (e.g., from about 0.01% to about 90%, from about 0.01% to about 80%, from about 0.01% to about 70%, from about 0.01% to about 60%, from about 0.01% to about 50%, from about 0.01% to about 40%, from about 0.01% to about 30%, from about 0.01% to about 20%, from about 0.01% to about 10%, from about 0.01% to about 5%, from about 0.01% to about 1%, from about 1% to about 99%, from about 5% to about 99%, from about 10% to about 99%, from about 20% to about 99%, from about 30% to about 99%, from about 40% to about 99%, from about 50% to about 99%, from about 60% to about 99%, from about 70% to about 99%, from about 80% to about 99%, from about 90% to about 99%, from about 10% to about 90%, from about 20% to about 80%, from about 30% to about 70%, from about 40% to about 60%, from about 10% to about 30%, from about 30% to about 50%, from about 50% to about 70%, or from about 70% to about 90%) of 4-AP and/or one or more derivatives of 4-AP.

In some cases, a composition described herein (e.g., a composition containing 4-AP and/or one or more derivatives of 4-AP) can include one or more pharmaceutically acceptable carriers (additives), excipients, and/or diluents. Examples of pharmaceutically acceptable carriers, excipients, and diluents that can be used in a composition described herein include, without limitation, saline (e.g., phosphate-buffered saline (PBS)), sucrose, lactose, starch (e.g., starch glycolate), cellulose, cellulose derivatives (e.g., modified celluloses such as microcrystalline cellulose and cellulose ethers like hydroxypropyl cellulose (HIPC) and cellulose ether hydroxypropyl methylcellulose (HPMC)), xylitol, sorbitol, mannitol, gelatin, polymers (e.g., polyvinylpyrrolidone (PVP), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A composition described herein (e.g., a composition containing 4-AP and/or one or more derivatives of 4-AP) can be administered to a mammal in need thereof (e.g., a mammal having hair loss) locally or systemically. In some cases, a compositions described herein can be administered locally. For example, a composition described herein can be administered locally by injection directly into, around, and/or near an area of hair loss on a mammal (e.g., a human). For example, a composition described herein can be administered locally by a topical administration onto, around, and/or near an area of hair loss on a mammal (e.g., a human). Compositions suitable for topical administration include, without limitation, creams, foams, gels (e.g., thermogels and cooling gels), balms (e.g., soothing balms), lotions, and ointments. In some cases, a topical composition containing 4-AP and/or one or more derivatives of 4-AP can be applied directly to the skin of a mammal (e.g., a human). In some cases, a topical composition containing 4-AP and/or one or more derivatives of 4-AP can be formulated as an after-shave. For example, 4-AP and/or one or more derivatives of 4-AP can be used to treat hair loss associated with shaving (e.g., hair loss associated with folliculitis caused by shaving).

In some cases, composition described herein (e.g., a composition containing 4-AP and/or one or more derivatives of 4-AP) can include one or more (e.g., one, two, three, four, five or more) additional agents that can be used together with (e.g., can be synergistic with) 4-AP and/or one or more derivatives of 4-AP to treat hair loss. The one or more additional agents that can be used together with 4-AP and/or one or more derivatives of 4-AP to treat hair loss can include any appropriate agent(s) used to treat hair loss. In some cases, an agent that can be used together with (e.g., can be synergistic with) 4-AP and/or one or more derivatives of 4-AP to treat hair loss can be an anti-inflammatory. In some cases, an agent that can be used together with (e.g., can be synergistic with) 4-AP and/or one or more derivatives of 4-AP to treat hair loss can be a disinfectant. In some cases, an agent that can be used together with (e.g., can be synergistic with) 4-AP and/or one or more derivatives of 4-AP to treat hair loss can be a hydrating agent. In some cases, an agent that can be used together with (e.g., can be synergistic with) 4-AP and/or one or more derivatives of 4-AP to treat hair loss can be an exfoliating agent. In some cases, an agent that can be used together with (e.g., can be synergistic with) 4-AP and/or one or more derivatives of 4-AP to treat hair loss can be a healing agent (e.g., can heal micro-lacerations in the skin).

In some cases, a composition described herein (e.g., a composition containing 4-AP and/or one or more derivatives of 4-AP) can be administered systemically. For example, a composition described herein can be designed for oral or parenteral (including intraperitoneal, subcutaneous, intramuscular, intravenous, and intradermal) administration to a mammal having hair loss. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition described herein can be formulated for parenteral administration (e.g., intraperitoneal injection or intravenous injection).

An effective amount (e.g., effective dose) of 4-AP and/or one or more derivatives of 4-AP in a composition described herein (e.g., a composition containing 4-AP and/or one or more derivatives of 4-AP) can vary depending on the severity of the hair loss, the route of administration, the age and general health condition of the subject, excipient usage, the possibility of co-usage with other therapeutic treatments such as use of other agents, and/or the judgment of the treating physician.

An effective amount of 4-AP and/or one or more derivatives of 4-AP in a composition described herein (e.g., a composition containing 4-AP and/or one or more derivatives of 4-AP) can be any amount that can treat hair loss on a mammal without producing significant toxicity to the mammal. An effective amount of 4-AP and/or one or more derivatives of 4-AP in a composition described herein can be any appropriate amount. In some cases, an effective amount of 4-AP and/or one or more derivatives of 4-AP in a composition described herein can be from about 0.05 milligrams per kilogram body weight (mg/kg) to about 1 mg/kg (e.g., from about 0.05 mg/kg to about 0.8 mg/kg, from about 0.05 mg/kg to about 0.6 mg/kg, from about 0.05 mg/kg to about 0.5 mg/kg, from about 0.05 mg/kg to about 0.3 mg/kg, from about 0.05 mg/kg to about 0.1 mg/kg, from about 0.1 mg/kg to about 1 mg/kg, from about 0.3 mg/kg to about 1 mg/kg, from about 0.5 mg/kg to about 1 mg/kg, from about 0.8 mg/kg to about 1 mg/kg, from about 0.1 mg/kg to about 0.9 mg/kg, from about 0.2 mg/kg to about 0.8 mg/kg, from about 0.3 mg/kg to about 0.7 mg/kg, from about 0.4 mg/kg to about 0.6 mg/kg, from about 0.1 mg/kg to about 0.3 mg/kg, from about 0.2 mg/kg to about 0.4 mg/kg, from about 0.3 mg/kg to about 0.5 mg/kg, from about 0.5 mg/kg to about 0.7 mg/kg, from about 0.6 mg/kg to about 0.8 mg/kg, or from about 0.7 mg/kg to about 0.9 mg/kg).

The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the hair loss may require an increase or decrease in the actual effective amount administered.

The frequency of administration of a composition described herein (e.g., a composition containing 4-AP and/or one or more derivatives of 4-AP) can be any frequency that can treat hair loss on a mammal without producing significant toxicity to the mammal. For example, the frequency of administration can be from about once a week to about once every two months, from about once every two weeks to about once every six weeks, or from about once every three weeks to about once a month (e.g., once every four weeks). The frequency of administration can remain constant or can be variable during the duration of treatment. A course of treatment with a composition described herein can include rest periods. For example, a composition described herein can be administered once a month over a six-month period followed by a rest period (e.g., a one or two month rest period), and such a regimen can be repeated multiple times. As with the effective amount, various factors can influence the actual frequency of administration used for a particular application. For example, the effective amount, duration of treatment, use of multiple treatment agents, route of administration, and severity of the hair loss may require an increase or decrease in administration frequency.

An effective duration for administering a composition described herein (e.g., a composition containing 4-AP and/or one or more derivatives of 4-AP) can be any duration that treats hair loss on a mammal without producing significant toxicity to the mammal. For example, the effective duration can vary from several days to several weeks, months, or years. In some cases, the effective duration for the treatment of hair loss can range in duration from about one month to about a lifetime. Multiple factors can influence the actual effective duration used for a particular treatment. For example, an effective duration can vary with the frequency of administration, effective amount, use of multiple treatment agents, route of administration, and severity of the hair loss being treated.

In some cases, the methods and materials described herein can be used as the sole active agent used to treat a mammal (e.g., a human) having hair loss. For example, a composition containing 4-AP and/or one or more derivatives of 4-AP can be used as the sole active agent(s) used to treat a mammal having hair loss.

In some cases, methods described herein also can include administering to a mammal (e.g., a human) having hair loss one or more (e.g., one, two, three, four, five or more) additional agents used to treat hair loss in addition to a composition described herein (e.g., a composition containing 4-AP and/or one or more derivatives of 4-AP). The one or more additional agents used to treat hair loss can include any appropriate agent(s) used to treat hair loss. Examples of agents that can be used to treat hair loss include, without limitation, minoxidil, finasteride, spironolactone (e.g., CaroSpir® and ALDACTONE®), dutasteride (e.g., AVODART®), and steroids (e.g., cortisone). In cases where a mammal having hair loss is treated with a composition described herein and is treated with one or more additional agents used to treat hair loss, the additional agent(s) used to treat hair loss can be administered at the same time or independently. For example, the additional agent(s) used to treat hair loss can be formulated into a composition containing 4-AP and/or one or more derivatives of 4-AP to form a single composition. In some cases, a composition described herein can be administered first, and the one or more additional agents used to treat hair loss can be administered second, or vice versa.

In some cases, methods described herein also can include can include subjecting a mammal (e.g., a human) having hair loss to one or more (e.g., one, two, three, four, five or more) additional treatments (e.g., therapeutic interventions) that are effective to treat hair loss. Examples of additional treatments that can be used as described herein to treat hair loss include, without limitation, hair transplant surgery, laser therapy (e.g., laser therapy using pulsed-dye lasers), electrical stimulation, scar massage, wearing pressure garments (e.g., compression socks), and silicone gel sheets. In some cases, the one or more additional treatments that are effective to treat hair loss can be performed at the same time as the administration of a composition described herein (e.g., a composition containing 4-AP and/or one or more derivatives of 4-AP). In some cases, the one or more additional treatments that are effective to treat hair loss can be performed before and/or after the administration of a composition described herein (e.g., a composition containing 4-AP and/or one or more derivatives of 4-AP).

The invention will be further described in the following examples, which do not limit the scope of the invention described in the claims.

### EXAMPLES

### Example 1: 4-AP Promotes Hair Regrowth and Generation

This Example demonstrates that 4-AP can increase hair regrowth and regeneration in mice in vivo.

### Procedure

Wild-type C57BL/6 mice (7-8 weeks old) were placed in the lateral decubitus position on a sterile paper sheet after anesthesia using intraperitoneal injection of ketamine (60 mg/kg) and xylazine (4 mg/kg). Sterile surgical instruments (autoclaved) were used during dorsal hair removal. The skin was prepared for hair removal on the dorso-lateral skin (from neck to tail) by shaving with a mechanical shaver, and then hair removal cream (Nair) was placed on the skin for 30-60 seconds. Hair was removed completely by gently wiping the skin with cotton balls soaked in warm water and disinfected using 70% ethanol wipes and betadine 3 times.

After dorsal hair removal, mice were placed in a clean cage on the heating pad for 30 minutes or until recovery. Immediately after recovery, mice were treated either with 4-AP (systemic, 10-50 μg/daily, intraperitoneal injection) or saline and then were returned to the animal facility.

Functional tests and tissue analyses were performed on mice for dorsal skin hair growth and then 4-AP was administered. 4-AP or saline was delivered systemically (ip) for 14 days after hair removal. All analyses were blinded.

The 4-AP treated and controls mice were subjected to functional analyses by dorsal mouse skin hair photography, and skin tissue was collected at day 14 for histology analysis and biomolecule analysis.

### Image Capturing

Mice were anesthetized during capturing of individual mouse dorsal photographs on sterile white background surface, and then dorsal area photographs were captured using a digital camera. The day when hair was removed was designated as day 0.

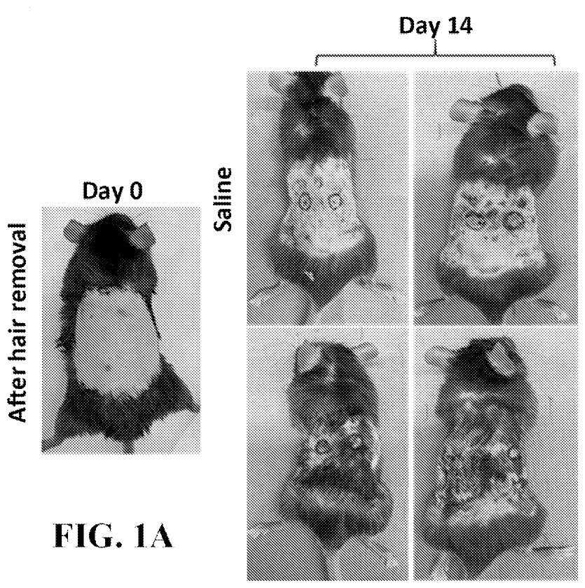

At day 14, the backs of the mice were photographed with a digital camera, to evaluate the darkening of skin color. The hair growth effect in each group was rated on a score ranging from 0 to 100%. In morphological observation (FIG. 1A and FIG. 1), hair growth area was determined as a percentage after photographing hair growth state on the backs of mice and observation was conducted after hair removal at day 0 and after 4-AP treatment (daily systemic) at day 14. No hair growth (and no pigmentation) was assigned the arbitrary value of 0. Skin darkening was given a value from 0 to 100%, with the higher number indicating darker skin/visible hair growth. The level of hair regrowth/pigmentation was quantified by the intensity of the darkness of the back skin in the same area.

Systemically administered 4-AP increased hair regrowth and regeneration in a standard model in rodents on the dorsal surface (FIG. 1A). 4-AP treatment led to around 50% hair regrowth and only 10% hair regrowth was observed in saline treated mice (FIG. 1A and FIG. 1i).

### Histological Observation of Skin Tissue

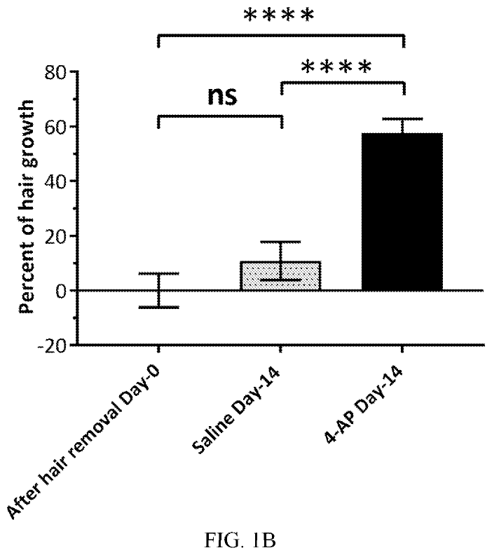

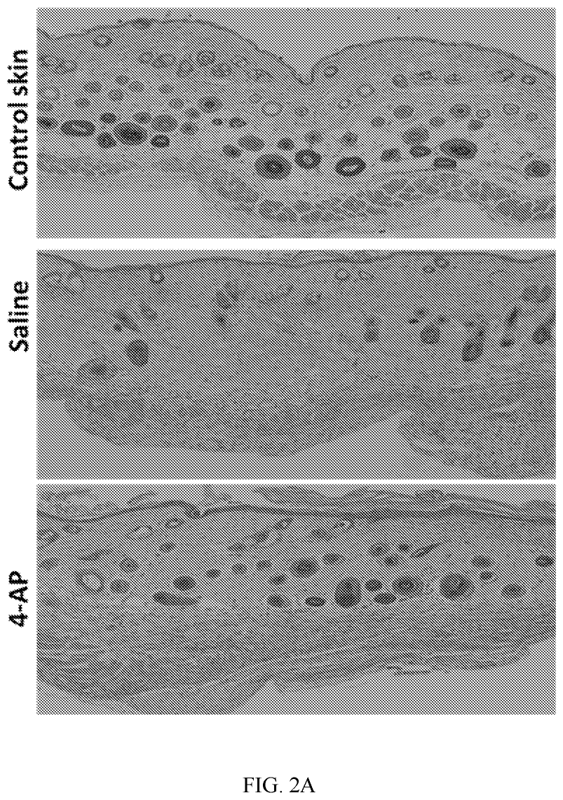

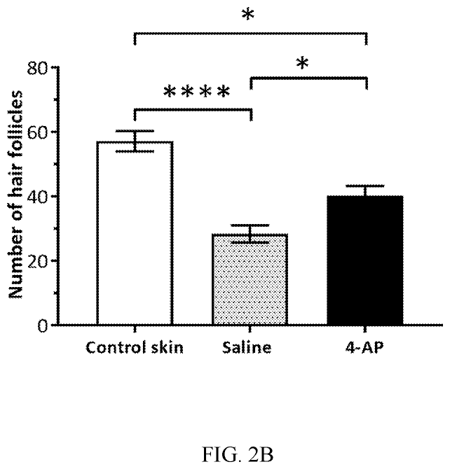

Mice were euthanized at day 14 and skin tissue was harvested. The dermal skin samples were fixed in 10% buffered formalin for 24 hours, followed by embedding in paraffin wax using standard techniques. General histology was visualized using hematoxylin and eosin (H&E) staining. The number of hair follicles was increased in the 4-AP treated mice compared with that in the saline treated mice in transverse sections on day 14 (FIG. 2A and FIG. 2B).

### Example 2: 4-AP Enhances Wound Healing, and Promotes Neurogenic-Mediator Production and Cutaneous Reinnervation

This Example demonstrates that 4-AP can accelerate skin wound healing by increasing cellular proliferation, migration, and de-differentiation, as well as neuro-mediator production.

### Results

### 4-Aminopyridine (4-AP) Promoting Wound Healing and Tissue Regeneration

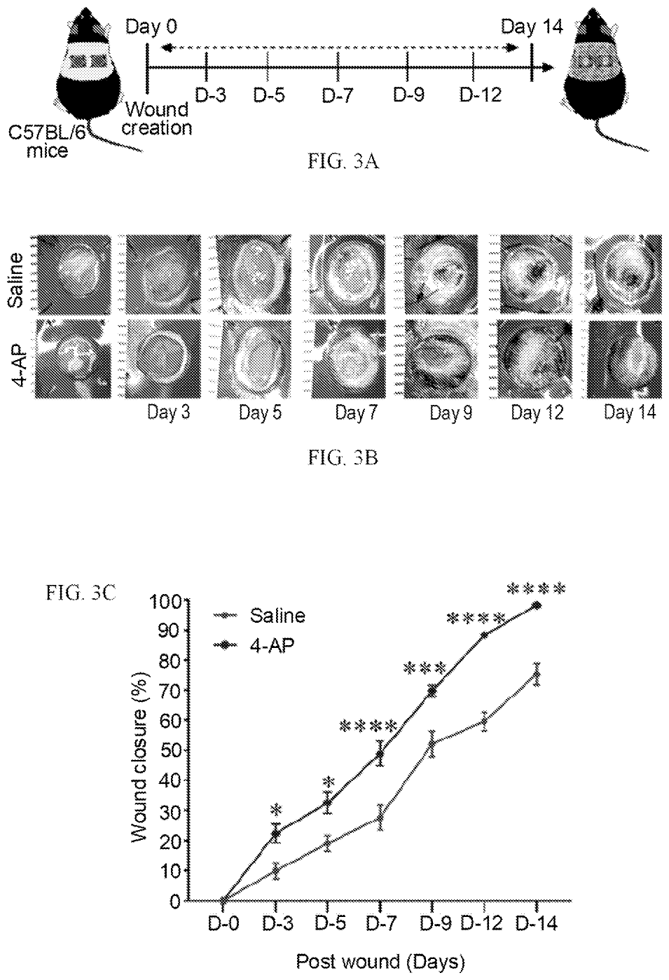

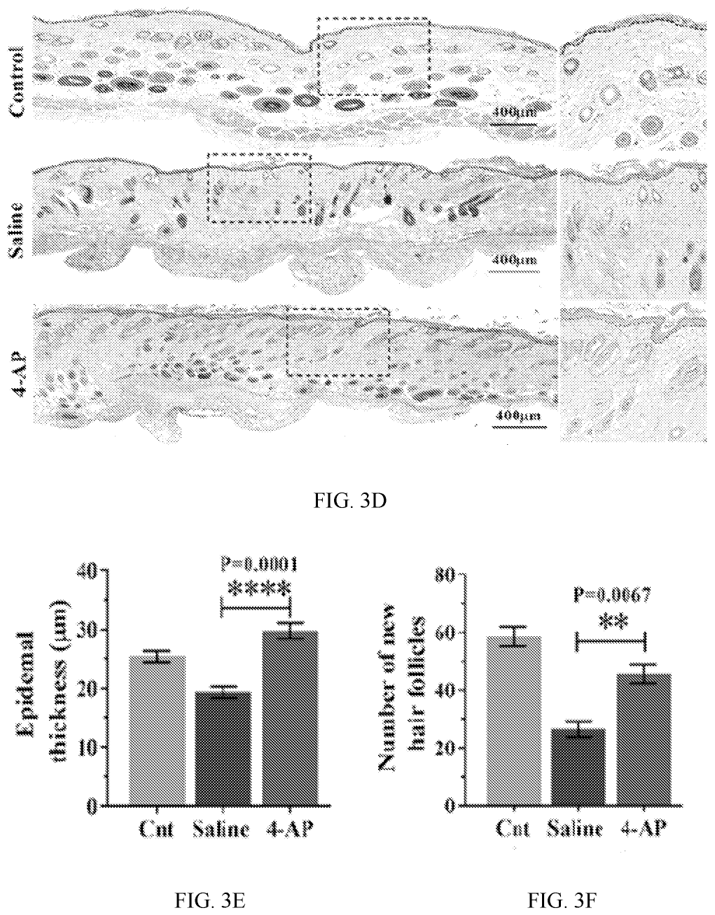

The effect of 4-AP in promoting cutaneous wound healing and tissue regeneration was investigated using a full thickness excisional wound model (FIG. 3A). Excisional wounds (5 mm) were splinted with silicone rings to prevent wound contraction, in 10-wk-old male C57BL/6 mice, which were then randomized and treated with either saline or systemic 4-AP (dosage to correspond to human dosage) daily for 14 days. Wounds were monitored by digital imaging for morphometry, percentage of wound healing, and tissue regeneration on days 3, 5, 7, 9, 12 and 14 post wound (PWD) (when tissue was harvested). Wound closure was significantly faster with 4-AP treatment from our first assessment point at day 3 onward (day 3: 22.35±0.30% vs. 9.88±0.28%; P<0.0199) to day 14 (day 14: 98.24±0.10% vs. 75.28±0.32%; P<0.0001) (FIG. 3B and FIG. 3C). By day 14, 4-AP treated mice had completely re-epithelialized and de novo hair follicles were present while saline treated mice had only 75% closure of the wound without evidence of hair follicle regeneration.

Tissue assessments were made on wound tissue harvested 14 days after wounds, when 4-AP treated mice had healed. Wound tissue was analyzed by morphometric analysis (hematoxylin and eosin (H&E) staining), which revealed newly formed epidermis within the healed wound that was significantly thicker in 4-AP treated mice (29.74±1.30 μM) compared to saline treated mice (19.38±0.96 μM) (FIG. 3D and FIG. 3E). Moreover, a significant increase in wound-induced hair neogenesis (WIHN) was evident in 4-AP treated mice when compared with saline-treated mice (FIG. 3D and FIG. 3F)—a feature known to correlate with skin regeneration (mean 45 hair follicles vs 25, 1.8 fold increase in WIHN; P=0.0067) (FIG. 3F).

### 4-AP Promotes Fibroblast Proliferation and Myofibroblast Formation Through TGF-β Signalling

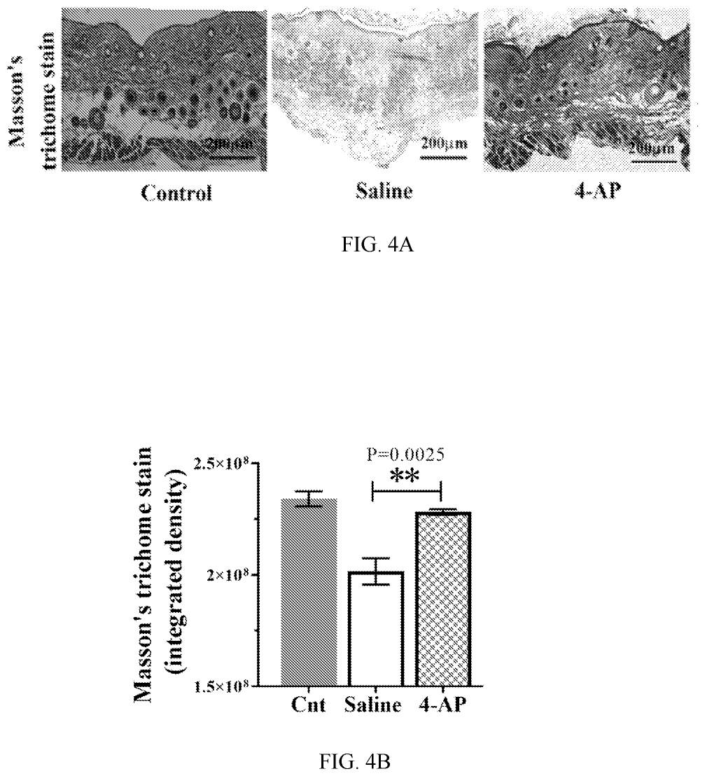

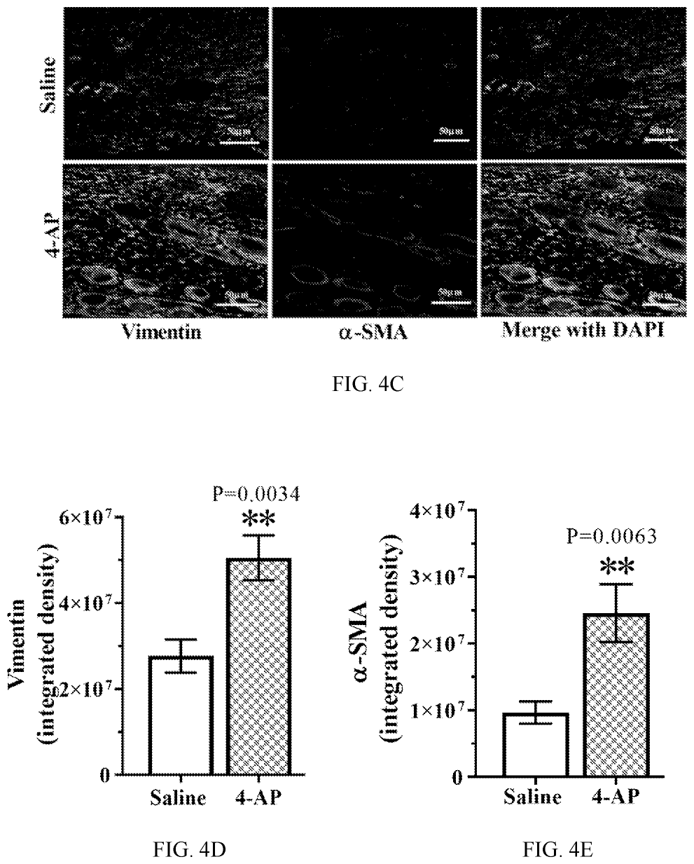

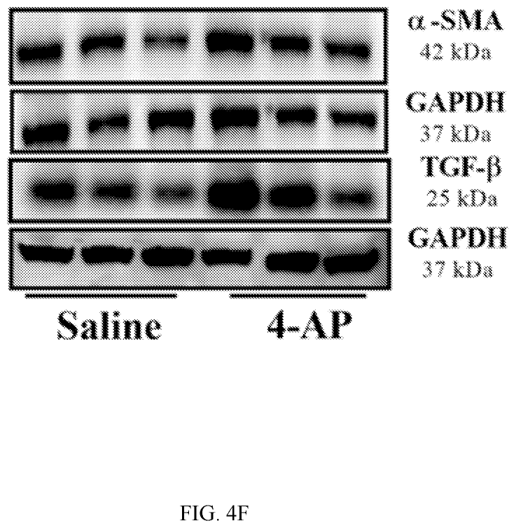

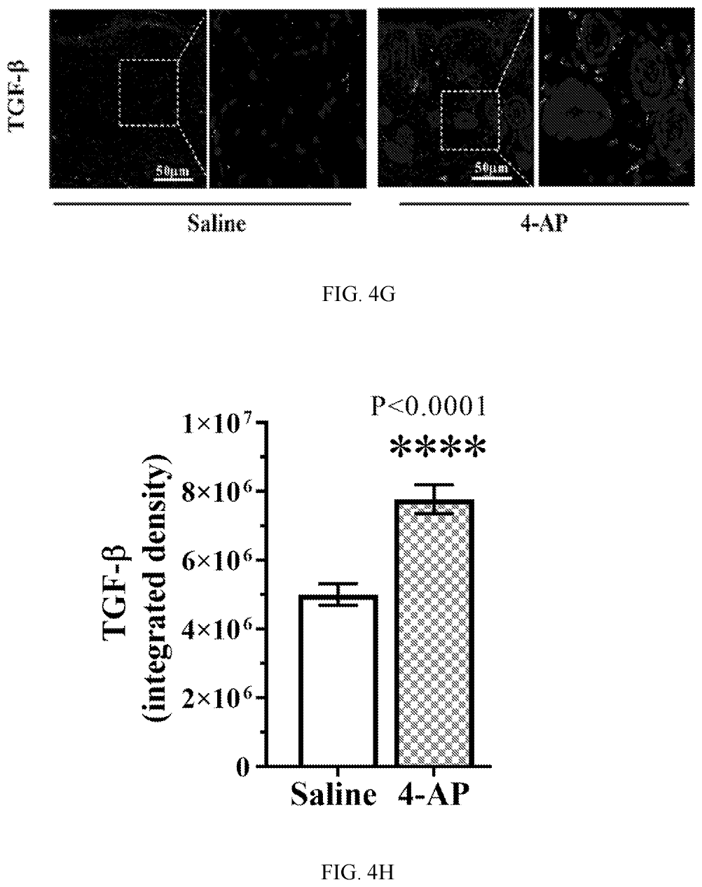

During wound healing, fibroblast migration and maturation play a significant role in the contraction, granulation, and proliferation phases. A key marker of fibroblast differentiation is α-smooth muscle actin (α-SMA) which signifies fibroblast differentiation into collagen-producing myofibroblasts. To test whether 4-AP treatment affects fibroblast maturation during wound healing, Masson's Trichrome staining was performed to measure collagen deposition in the healing wound. Staining revealed elevated collagen deposition in dermis from 4-AP treated mice compared to saline (FIG. 4A and FIG. 4B).

Immunohistochemistry for vimentin (a fibroblast marker) and α-SMA (FIG. 4C-FIG. 4E) revealed more vimentin-positive fibroblasts and α-SMA-positive myofibroblasts in wound tissue from 4-AP treated mice than saline treated mice, which was consistent with western blot (WB) analysis of α-SMA expression (FIG. 4F and FIG. 4G). Transforming growth factor-β (TGF-0) is known to be involved in fibroblast to myofibroblast differentiation, and significant increases in TGF-β protein expression were found with 4-AP treatment compared to saline treatment (FIG. 4F, FIG. 4H, FIG. 4I, and FIG. 4J).

### 4-AP Induces Neo-Angiogenesis in Granulation Tissue and Enhances Wound Healing

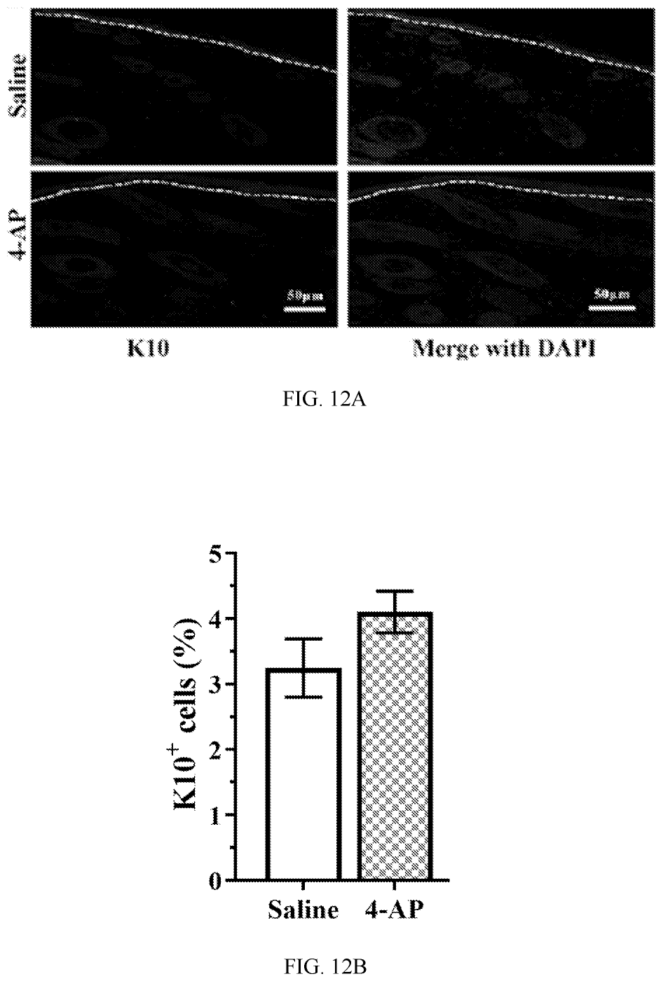

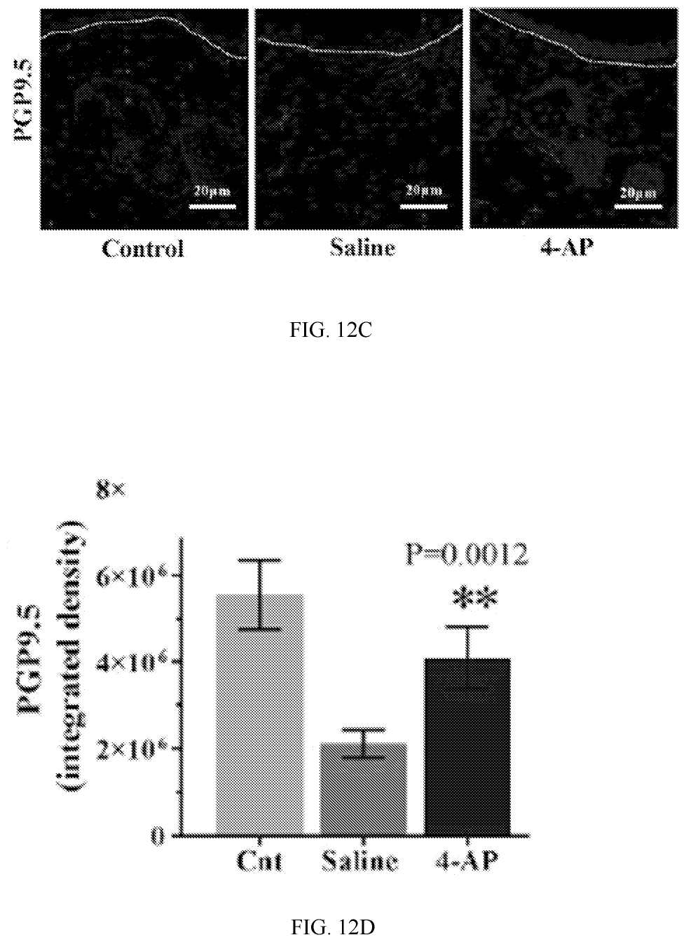

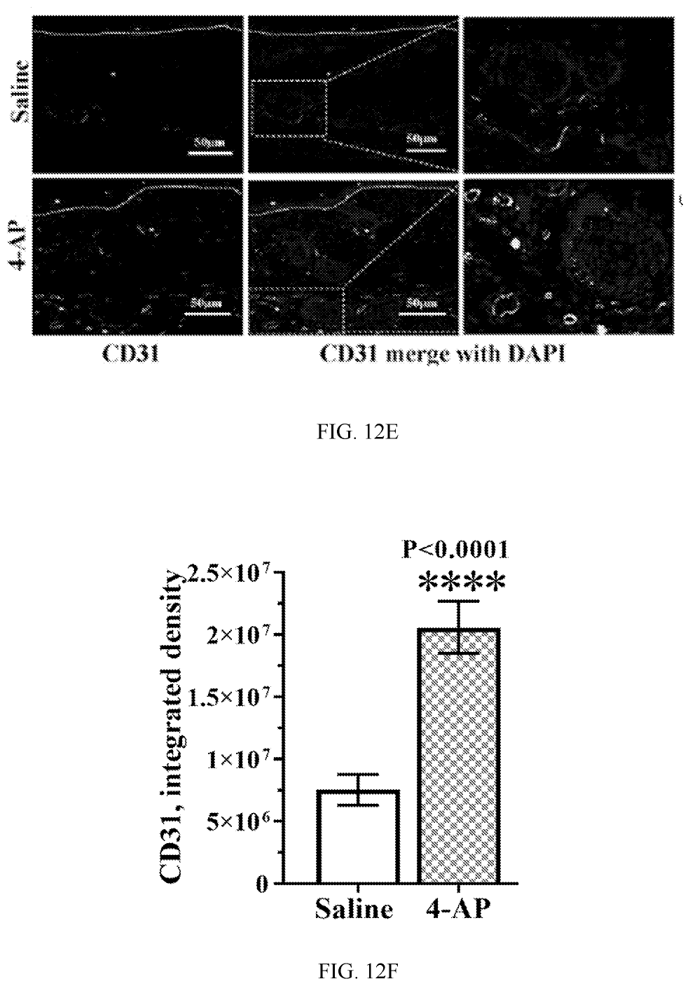

Neo-angiogenesis is necessary to provide nutrients and oxygen to healing wounds. To assess the difference in wound neo-vascularization with 4-AP treatment, immuno-fluorescence staining for the endothelial specific marker CD31 was performed. Larger and more abundant blood-vessel networks were observed in dermal tissue from 4-AP treated mice than saline treated mice (FIG. 12E). The quantification of CD31 intensity revealed statistically significant increases in blood vessels with 4-AP treatment compared to saline treatment (FIG. 12F). These results suggest 4-AP treatment enhanced proliferation of endothelial cells to induce neo-angiogenesis—a likely factor in improved wound healing and tissue regeneration.

### 4-AP Increases Keratinocyte Proliferation, Migration and Epithelial Stem-Cell Markers During Wound Healing

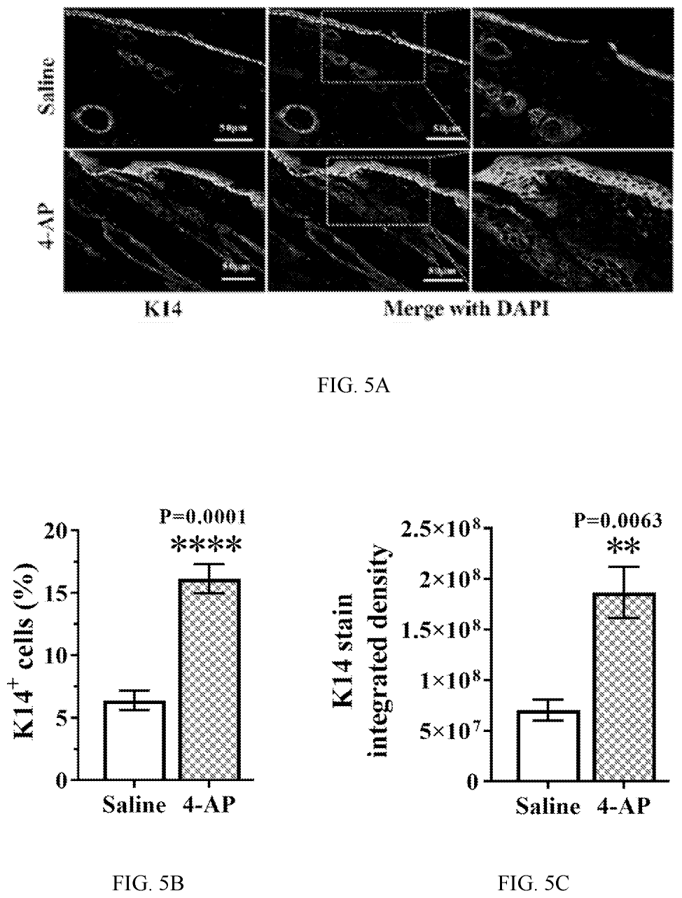

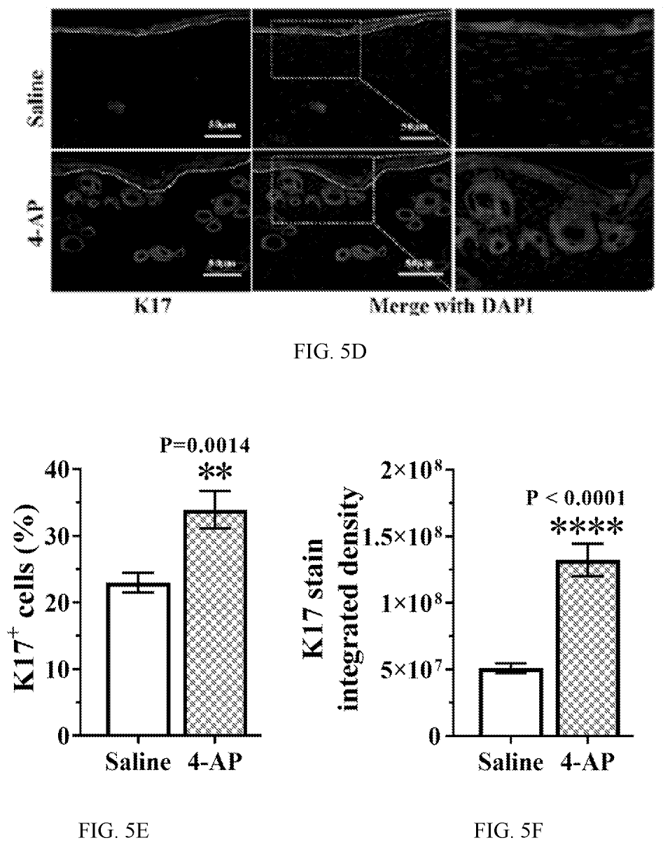

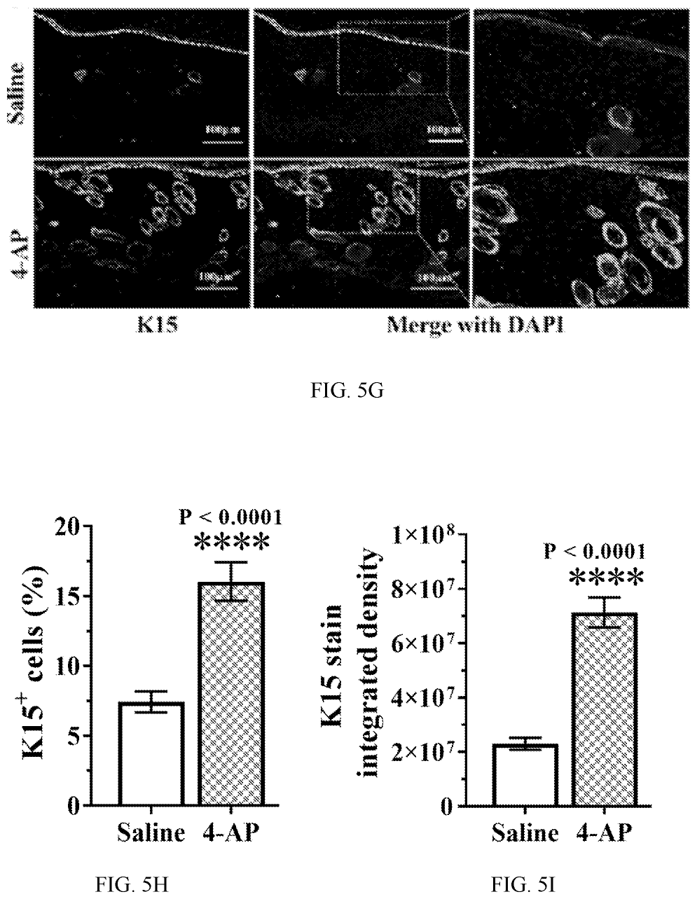

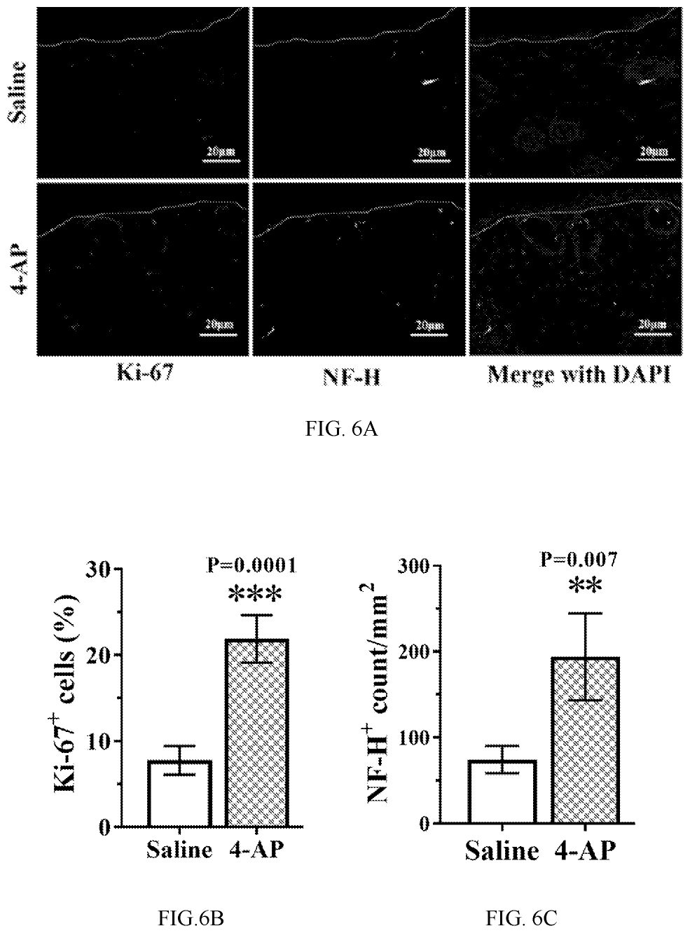

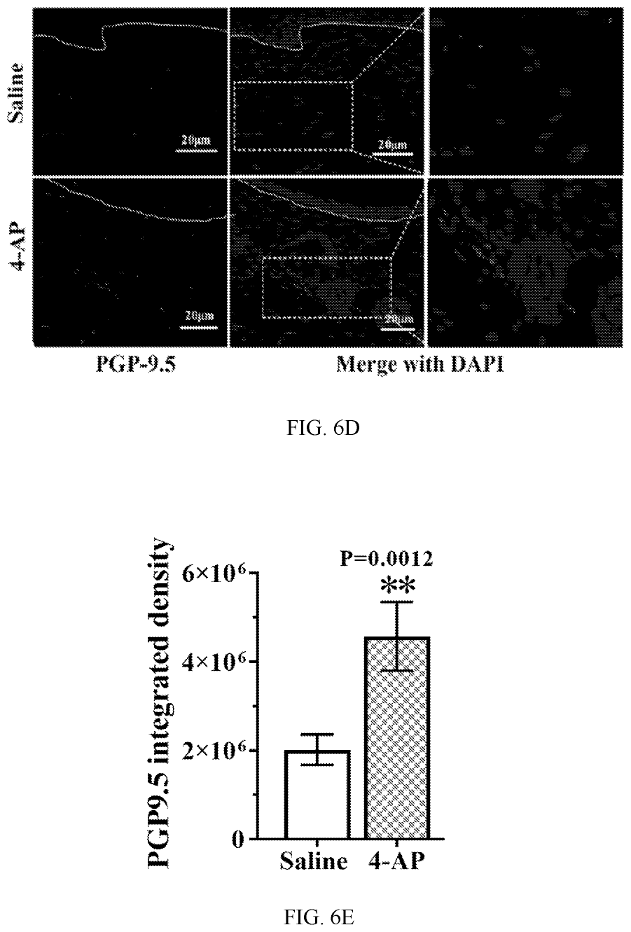

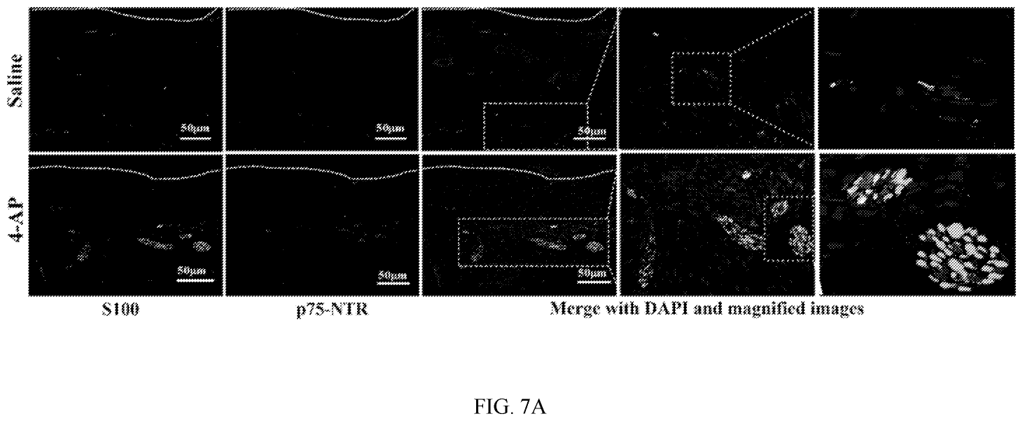

Re-epithelialization involves keratinocyte proliferation, and is marked by expression of the basal keratinocyte proliferation marker, Keratin 14 (K14). Significant increases in K14⁺ keratinocytes were observed in the epidermis and in de novo hair follicles in wound beds of 4-AP treated mice (FIG. 5A) compared with saline-treated mice (2.5 fold increase: 13.72±1.403% vs. 5.731±1.189%) (FIG. 5B and FIG. 5C). This was also found in the expression of keratin 17 (K17), a wound-induced hair follicle associated keratin (FIG. 5D-FIG. 5F), and corresponded well to expression for the proliferation marker Ki67, which was used in conjunction with staining for neurofilament-H (NF-H) (see below, FIG. 6A). These findings were consistent with findings of increased dermal hyperplasia (FIG. 3B and FIG. 3C), and hair follicles necessary for faster re-epithelialization and healing in 4-AP treated mice. Cutaneous wound healing is regulated by hair-follicle regeneration, which in turn is related to the differentiation and activation of SKPs towards a committed phenotype. Histo-morphometric analysis of healing tissue from 4-AP treated mice had more hair follicles than saline treated controls (FIG. 3D and FIG. 3F). This was also true for the hair-follicle stem cell marker keratin 15 (K15) (FIG. 6A) (two fold increase in K15+ cells) (FIG. 6B). 4-AP stimulates hair follicle and keratinocyte proliferation and SKP activation.

### 4-AP Promotes Reinnervation and Neuropeptide Expression

Whether the role of 4-AP on myelination and regeneration relates to wound healing through improved reinnervation was examined. The proliferation marker Ki-67 was used in conjunction with the neuronal marker NF-H, and significantly increased co-staining was revealed in mice treated with 4-AP treatment compared to saline treated controls. Ki-67⁺ in hair follicle bulges and epidermis was increased 2-fold (21.87±2.763 cells/mm² vs. 7.754±1.664% cells/mm²) (FIG. 6C and FIG. 6D) and NF-H axonal counts were increased 2.5 fold (194±50.51 count/mm² vs. 74.19±15.85 count/mm²) in 4-AP treated mice compared with saline treated controls (FIG. 6C and FIG. 6E). Moreover, NF-H stained axons in the 4-AP treated mice were more often encountered in direct association with Ki-67⁺ hair follicles (FIG. 6C) than in saline treated controls—an important qualitative finding given that hair follicles are known to be associated with sympathetically innervated arrector pili muscles. Thus, the association between nerve function and hair follicle stem cells under the influence of 4-AP supports reinnervation as a possible factor in the formation of de novo hair follicles during wound healing.

To more completely assess 4-AP's effects on neuropeptides and neuronal modulation in wound healing, 4-AP's effect on protein gene product 9.5 (PGP 9.5), which is also known to promote wound healing, was assessed. Fourteen days post wound, PGP 9.5⁺ nerve fibres in the healed wounds were twice as abundant in 4-AP treated mice (4096938±713297 integrated density/mm²), than in saline treated mice (2107970±325039 integrated density/mm²) (FIG. 6F and FIG. 6G). The levels of PGP 9.5 in 4-AP-treated mice were not significantly different from seen in uninjured tissue (FIG. 12C and FIG. 12D). This suggests that 4-AP increases expression of neuropeptide mediators in wound healing.

### 4-AP Induces Schwann Cell (SC) Proliferation and De-Differentiation

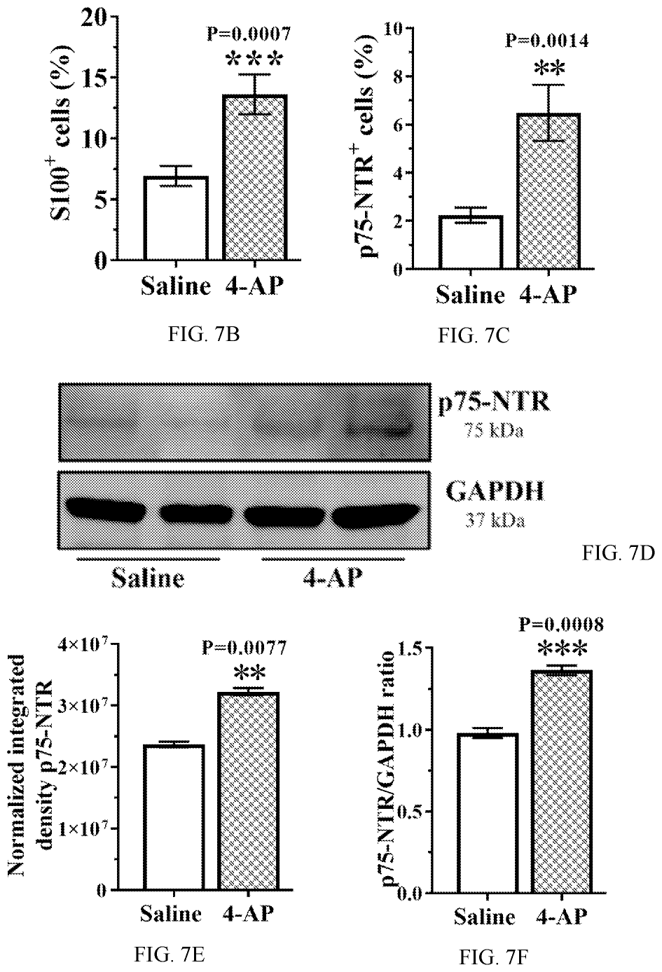

Schwann cells (SC) are critical players in wound healing and are associated with axons around hair-follicles in the wound bed. In the setting of injury, SCs de-differentiate to a non-myelinating state and begin to secrete neurotrophins like NGF, a state marked by expression of p75-NTR. Using immunohistochemistry for two different markers of SCs (S100, a pan SC marker and p75-NTR, a marker of SC de-differentiation), it was found that SCs were located within the hypodermis and dermis of the healed wounds (FIG. 7A), and that the density of S100⁺ cells around nerve bundles was far greater in 4-AP treated mice than in saline treated controls (FIG. 7A) (3-fold increase: 6.483±1.163% vs. 2.242±0.3159% positive cells) (FIG. 7B). With 4-AP treatment, these SCs were more likely to be in the de-differentiated, growth-factor elaborating state, as evidenced by increased numbers of double positive (S100⁺, p75-NTR⁺) SCs in wound tissue from 4-AP treated mice compared with saline treated mice (FIG. 7A-FIG. 7C) and confirmed by WB for p75-NTR (FIG. 7D and FIG. 7E).

### 4-AP Treatment Enhances SC Neural Transcription Factor Expression and Neuromediator Synthesis

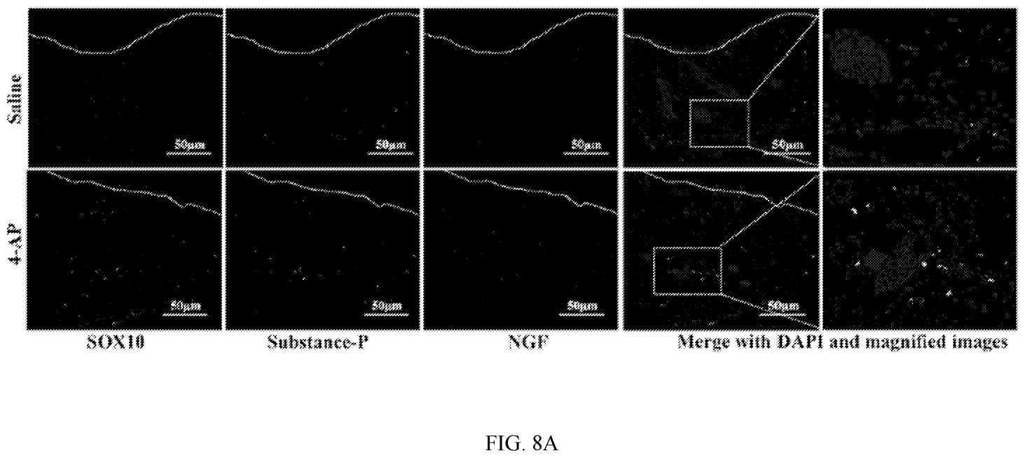

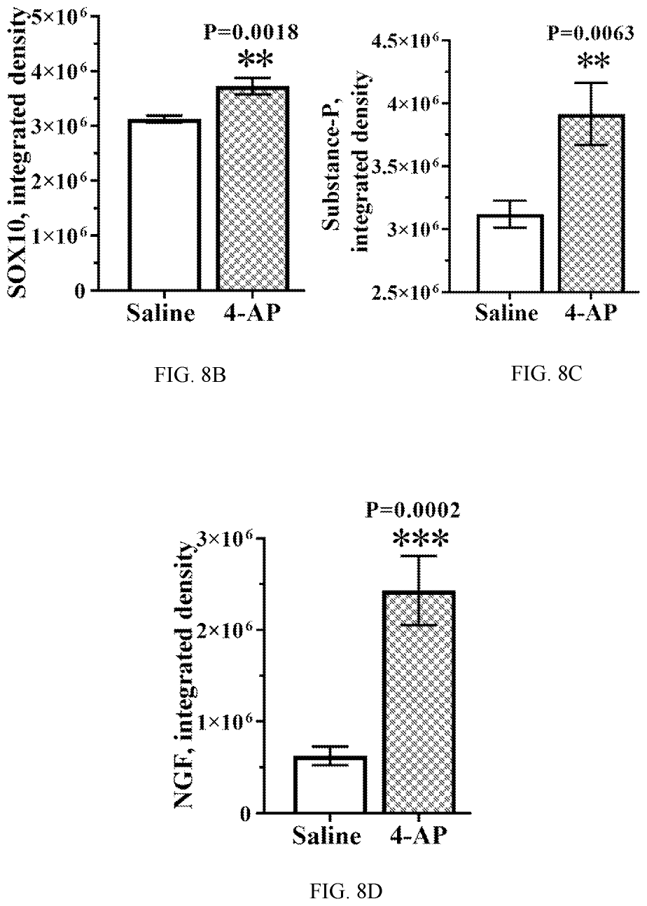

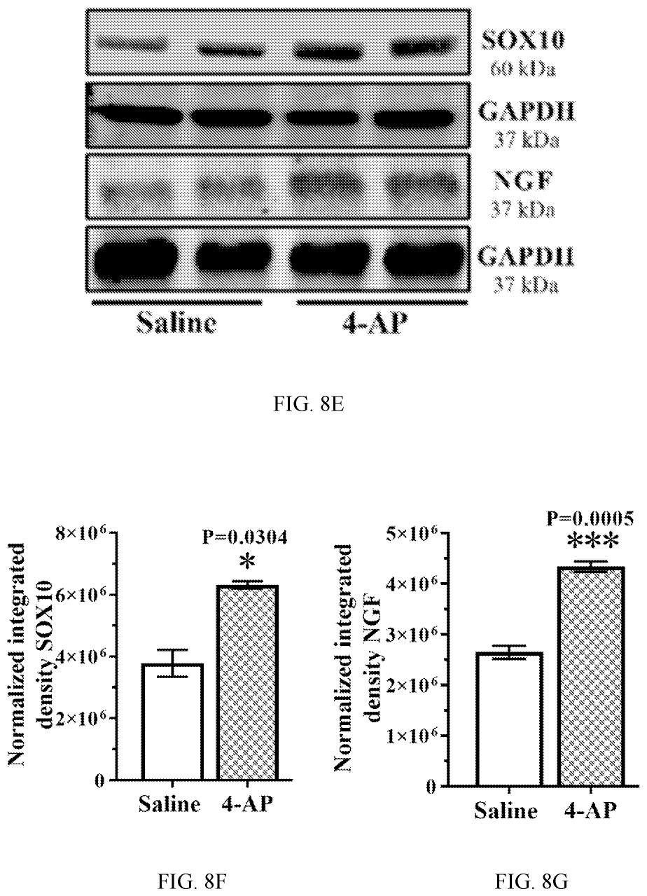

SC originate from migratory neural crest cells (NCCs) that express SOX10, which is required for myelin production. Elevated SOX10 expression promotes conversion of mesenchymal cells into p75-NTR expressing neural crest stem cells (NCSC) and depletion of SOX10 expression significantly delays wound healing and tissue regeneration. NGF and its receptor, p75-NTR, play significant role in the wound healing process by inducing nerve sprouting from injured nerve endings. NGF also acts on non-neuronal cells to sensitize them to substance-P, which in turns further stimulates more NGF secretion ensuring that keratinocytes, for example can elaborate and respond to neuronal factors along with neurons. Significantly increased SOX10, substance-P, and NGF (NGF 4-fold increase: 2429872±375280 integrated density/mm² vs. 626967±101856 integrated density/mm²) expression was found in tissue harvested from 4-AP treated mice compared to saline treated mice (FIG. 8). These factors, which are known to be associated with both nerve regeneration in the wound bed and accelerated healing were increased within the wound with 4-AP treatment.

### 4-AP Effectively Stimulates Proliferation and Migration in Primary Cultures of Human Skin Derived Primary Cells In-Vitro

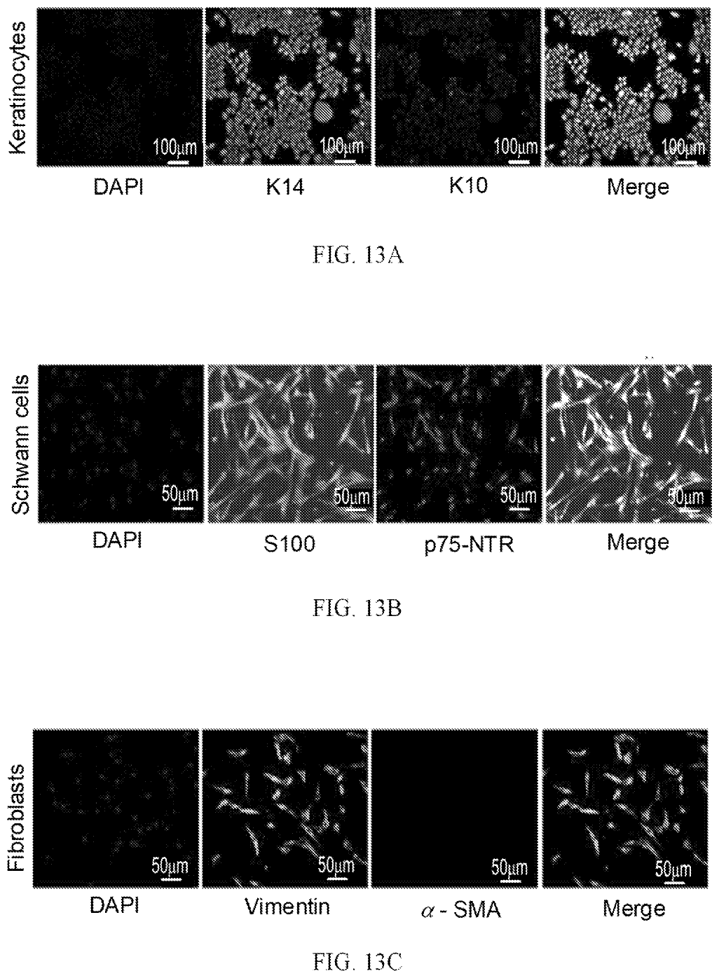

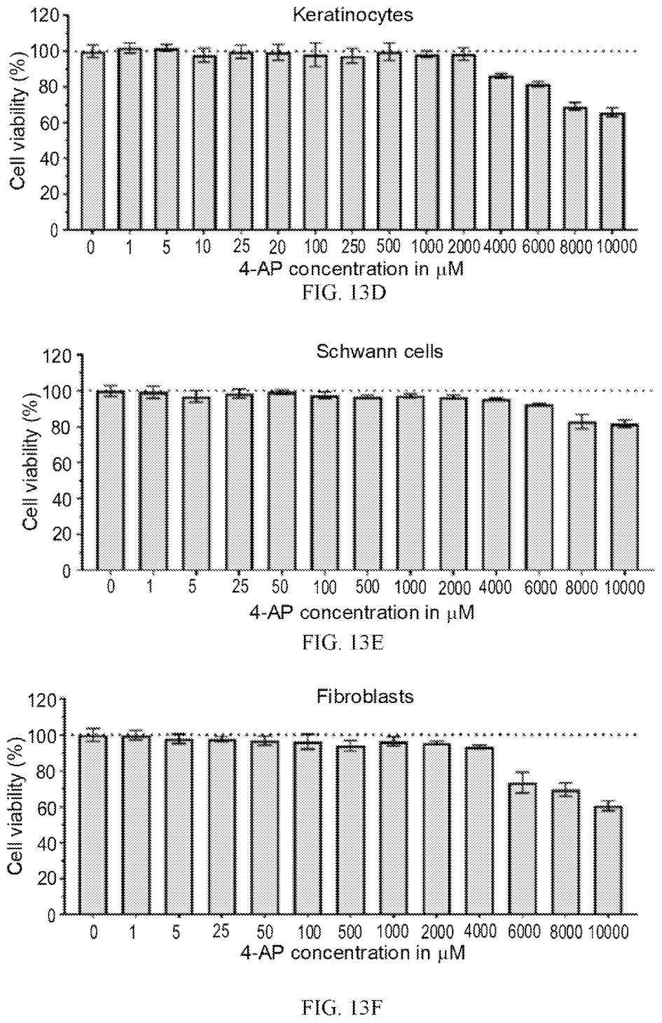

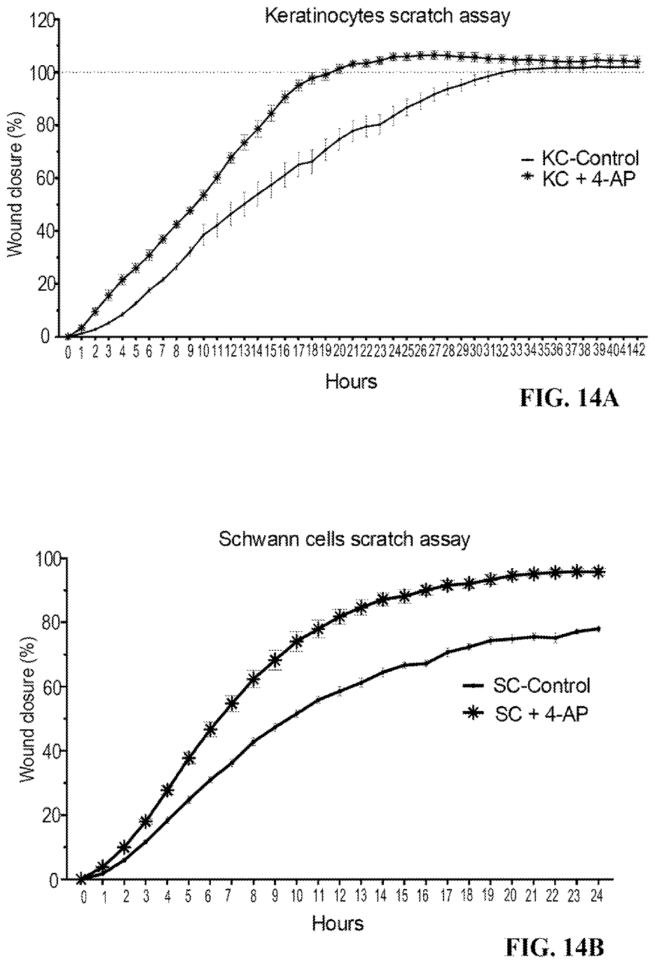

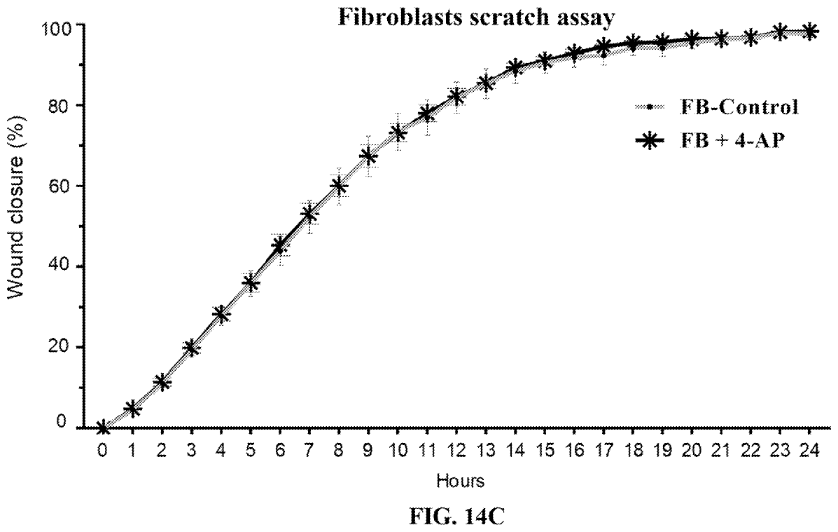

As described above, 4-AP accelerates wound closure and enhances WIHN, angiogenesis and nerve regeneration in healed wound. To further understand the specific effects of 4-AP on skin cells, primary, normal human epidermal keratinocytes (NHEKs), fibroblasts, and dermal SCs were cultured in the presence of 4-AP. The purity and identity of each cell type was confirmed with immunohistochemistry for characteristic markers (FIG. 13A-13C) and determined cell viability was not affected by concentrations of 4-AP used in vitro as described elsewhere (Manoukian et al., J. Control Release, 296:54-67 (2019)) (FIG. 14A-FIG. 14C). Automated wound scratch assays were performed on confluent monolayers of keratinocytes, fibroblasts, and SCs, with and without 1 mM of 4-AP treatment.

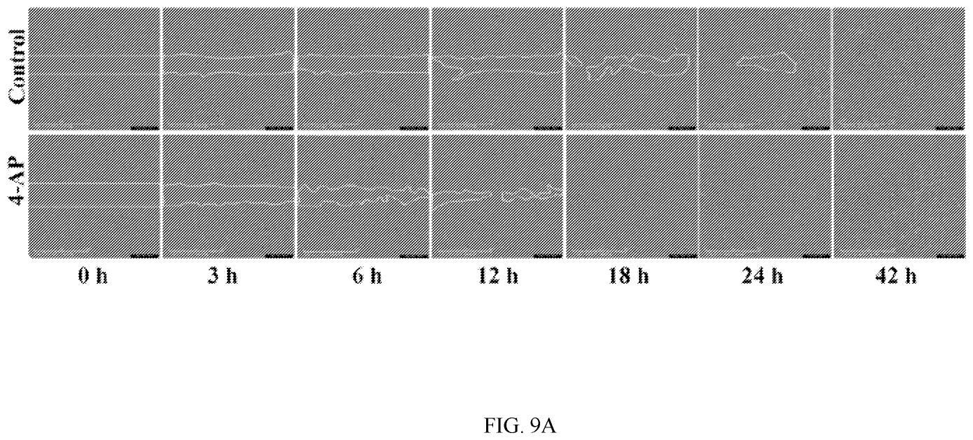

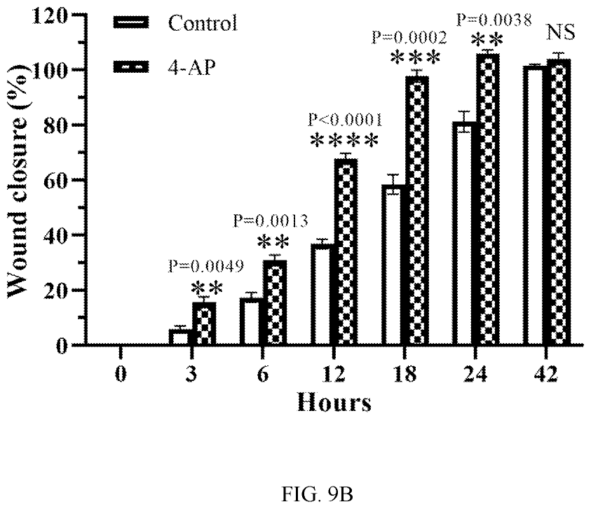

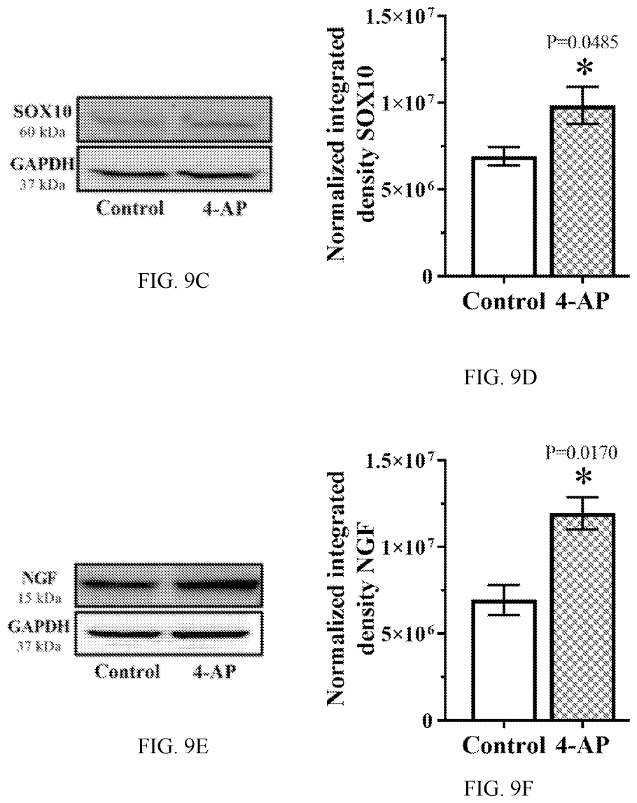

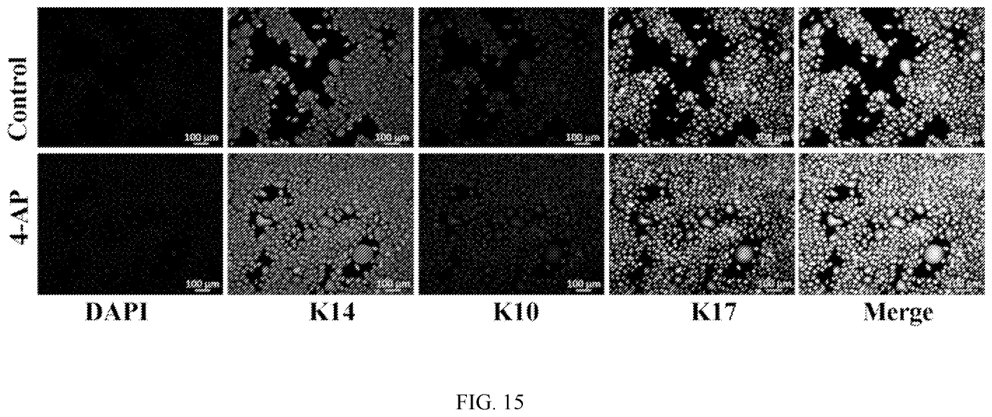

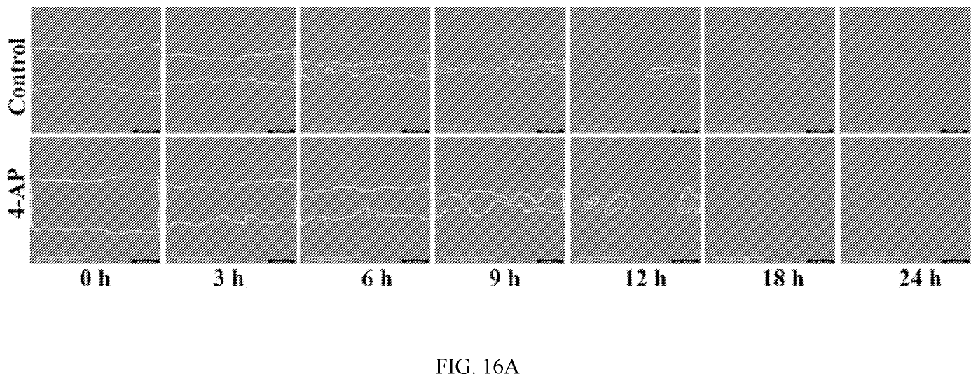

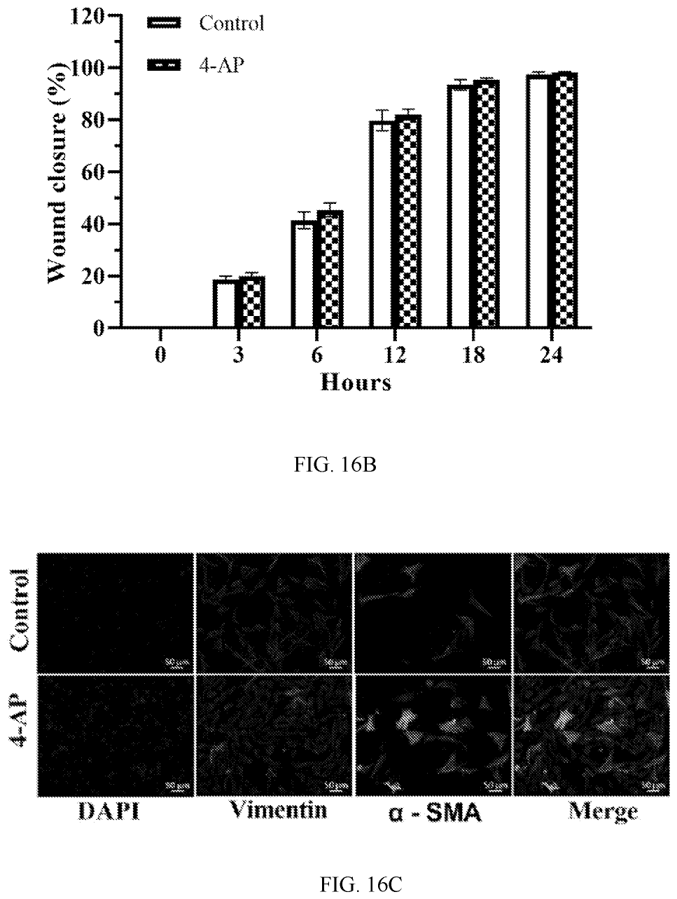

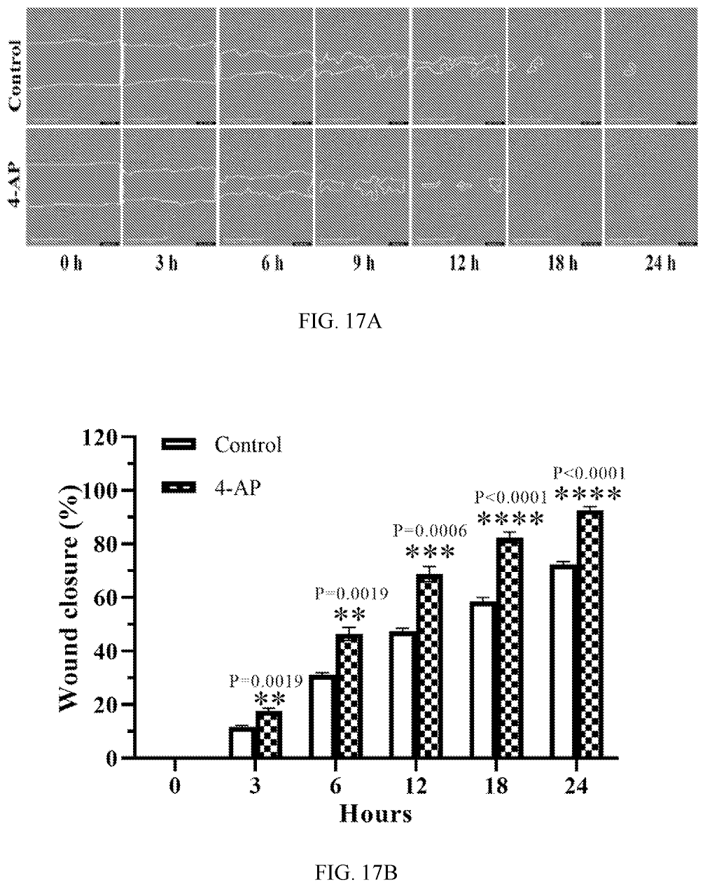

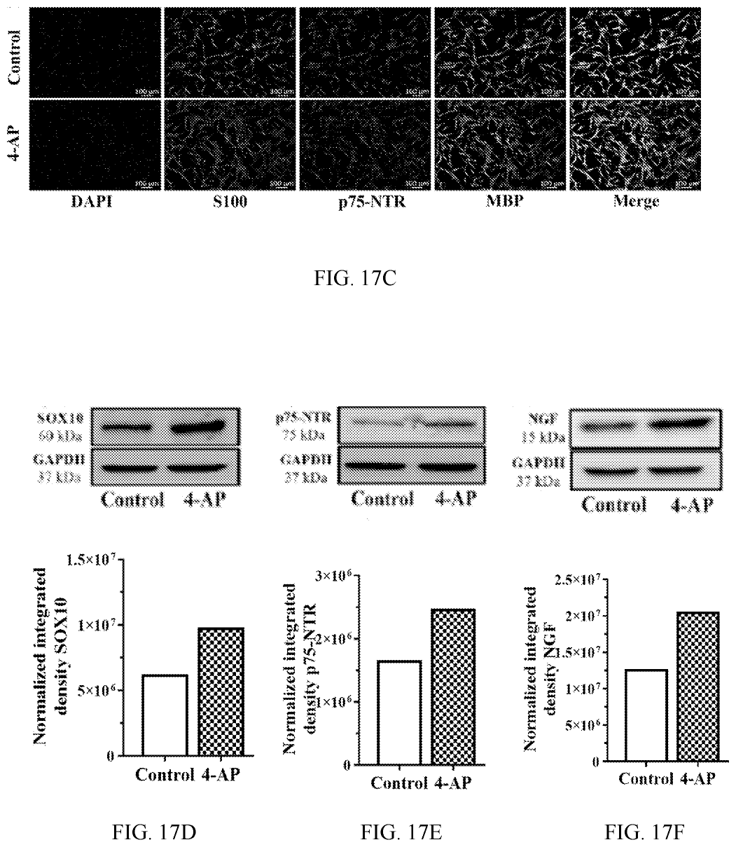

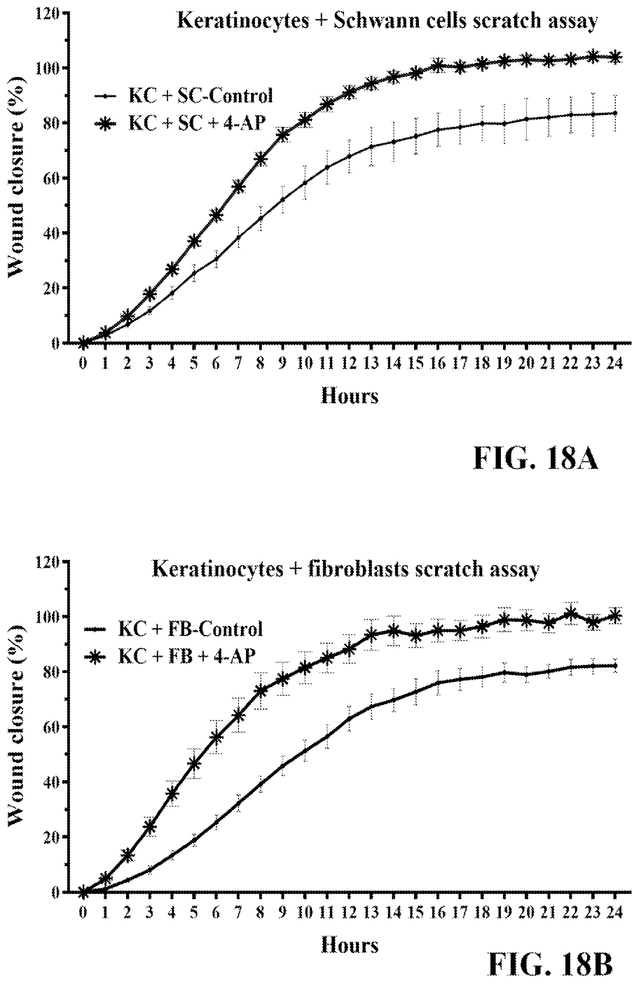

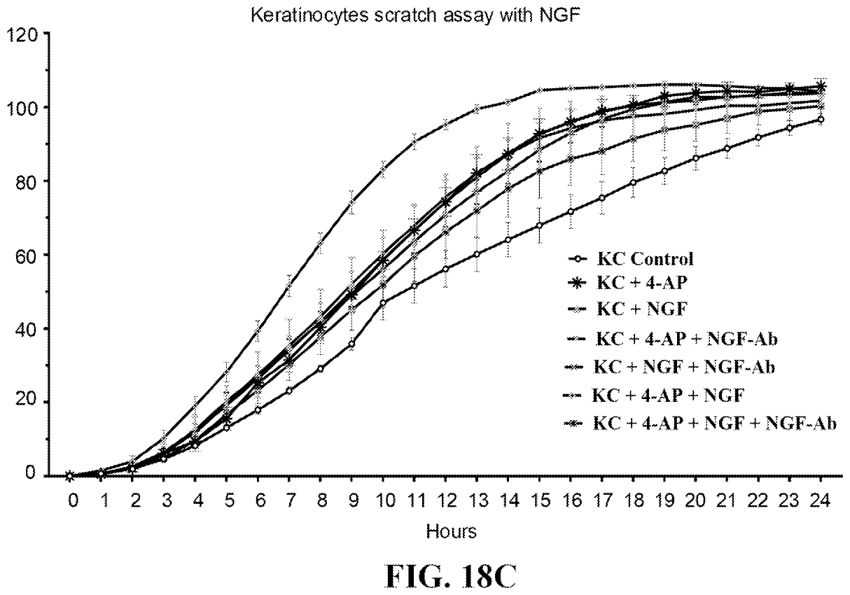

4-AP treatment accelerated scratch closure and keratinocyte migration (FIG. 9A and FIG. 14A) as soon as 3 hours, with complete scratch closure occurring at 18 hours, in contrast to control cultures without 4-AP that closed at 32 hours (FIG. 9A and FIG. 9B). SOX10 and NGF expression in 4-AP treated keratinocytes were both increased (FIG. 9C-FIG. 9F) as was the expression of keratin proteins associated with basal, proliferating keratinocytes (K14 and K17). 4-AP did not increase the expression of keratins associated with keratinocyte differentiation (K10) (FIG. 15), suggesting that 4-AP promotes a more proliferative, stem cell-like phenotype in keratinocytes, that is necessary for accelerated scratch closure. In contrast, fibroblast migration was unaffected by 4-AP, but 4-AP did induce a conversion of fibroblasts to myofibroblasts with increased vimentin and α-SMA protein expression (FIG. 14B and FIG. 16A-16C). In SCs, 4-AP accelerated scratch closure was increased (80% by 11 hours with 4-AP treatment compared with 23 hours without treatment) (FIG. 14C and FIG. 17A-17B), and 4-AP increased SOX10 and NGF expression compared to controls (FIG. 17C-17E). Effects on keratinocytes, fibroblasts and SCs reflect improvements associated with 4-AP that are reminiscent of those found in-vivo.

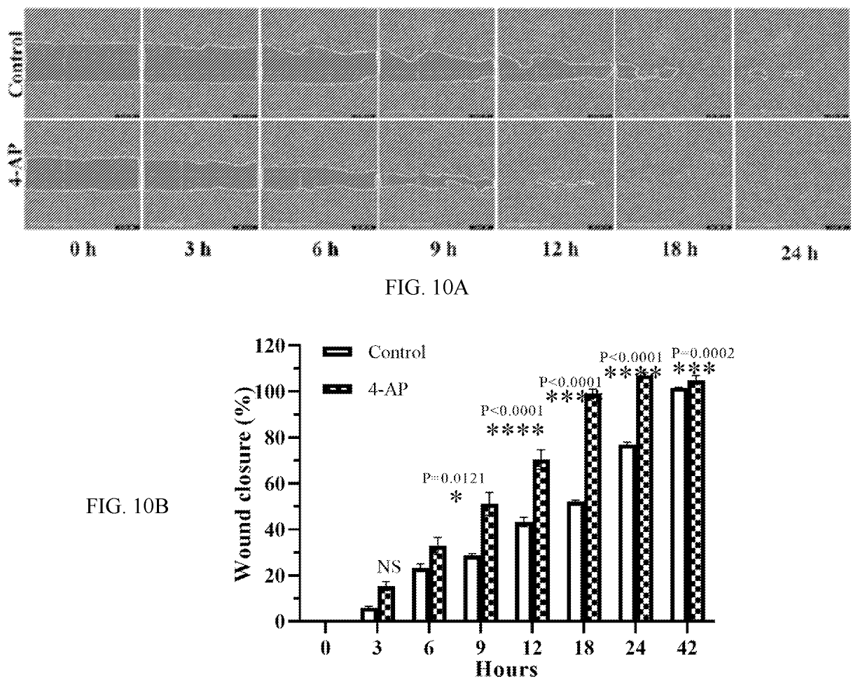

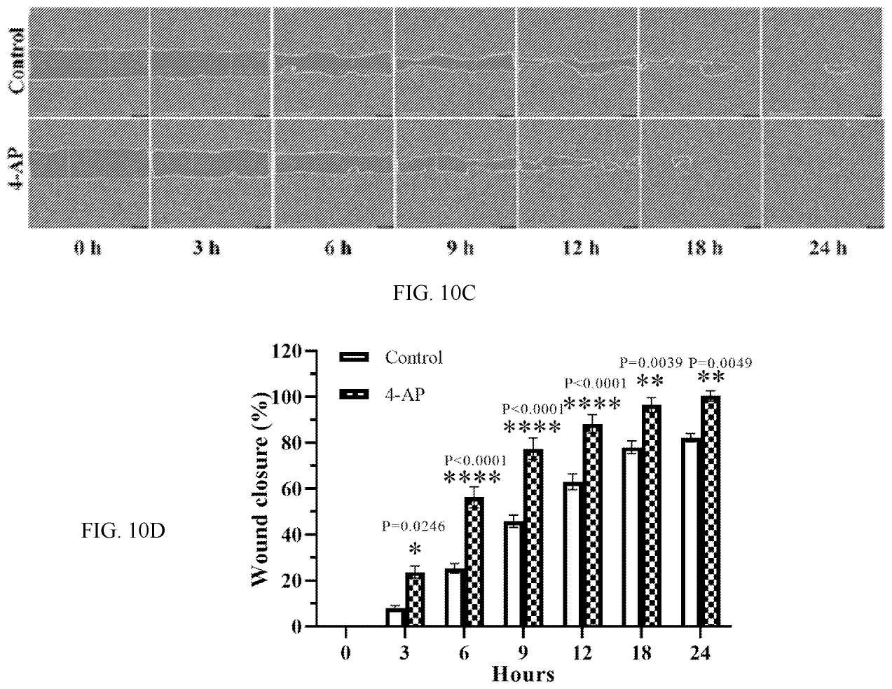

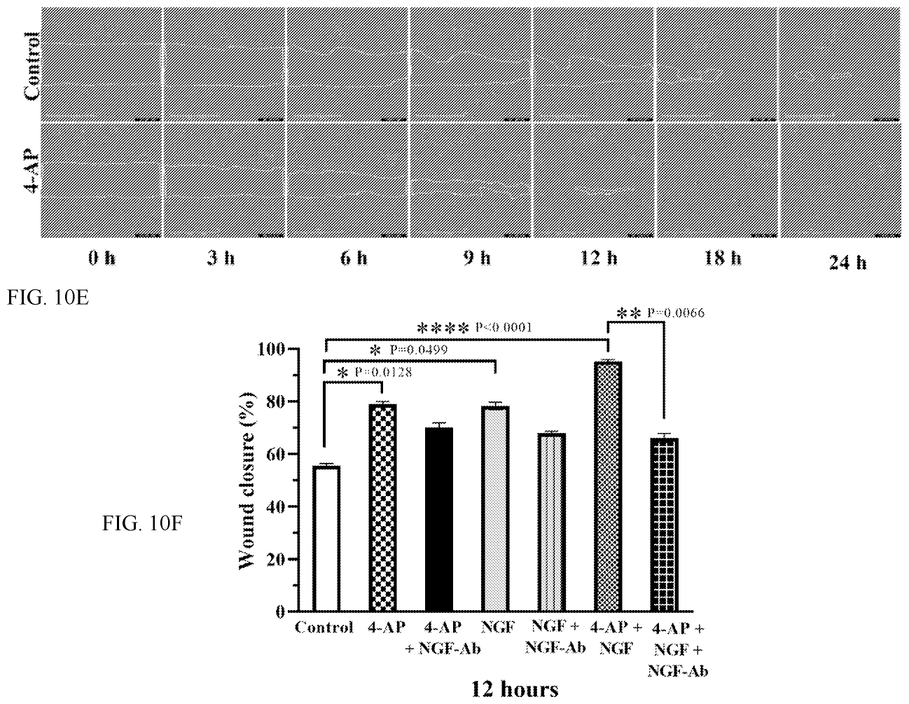

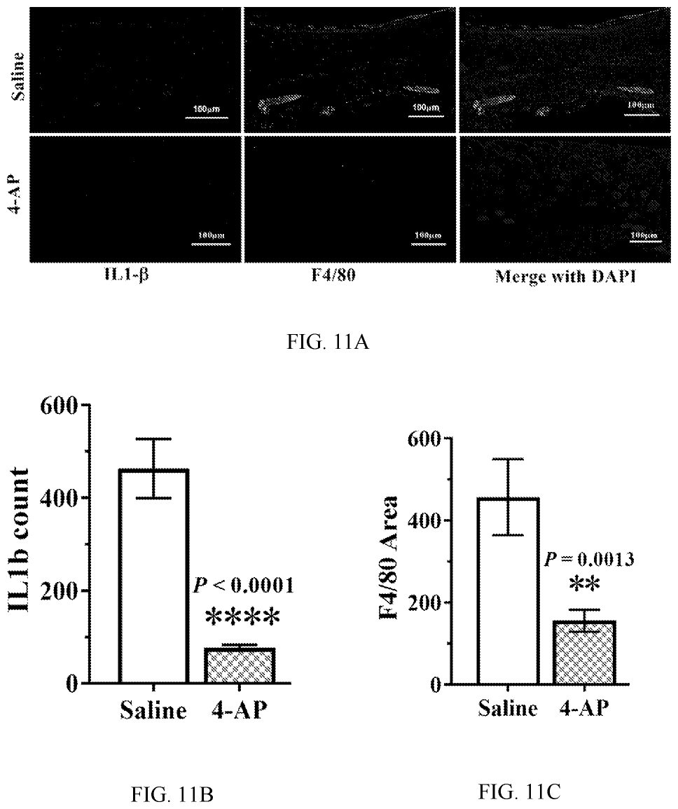

Given that keratinocytes, fibroblasts and SCs cells all interact during wound healing in-vivo, co-culture experiments were conducted with pairs of cell types with and without 4-AP treatment to determine if 4-AP promotes synergistic properties. Co-culturing cells accelerated scratch culture closure rates in general. However, keratinocytes co-cultured with SCs in a ratio mimicking that of actual epidermal skin (10:1 keratinocytes:SCs) closed a scratch within 15 hours with 4-AP treatment, which was significantly faster than scratch closure without treatment, which took 20 hours. At 15 hours, the percentage of wound closure was at 98% in 4-AP treated but only 80% in non-treated scratch cultures (FIG. 10A and FIG. 10B). This observation confirms that the addition of SCs alone can enhance keratinocyte migration and scratch closure, and that 4-AP further accelerates closure beyond the addition of SCs.

In co-culture scratch assays of keratinocytes with fibroblasts (ratio 10:1), similar effects on migration and scratch closure were found (FIG. 10C and FIG. 10D). The acceleration found with this particular pair of cells in co-culture was not unique, as it was also found that 4-AP treatment led to faster scratch closure in keratinocytes co-cultured with fibroblasts (ratio, 10:1) (measured at 15 hours: 93% in 4-AP treated vs. 72% in non-treated) (FIG. 10C and FIG. 10D).

### The Relationship Between NGF and 4-AP Treatment in Enhanced Keratinocyte Proliferation and Migration In Vitro.

Since 4-AP, NGF, and electrical stimulation are approved for use in various conditions, it was sought to determine if NGF is a factor mediating 4-AP induced scratch closure. Keratinocyte scratch closure cultures were treated with 4-AP, NGF, or a NGF neutralizing antibody, either alone or in combination. Treatment of keratinocyte cultures with either 4-AP or NGF accelerated scratch closure, with 75% vs 56% in control cultures at 12 hours. NGF-neutralizing antibody impaired closure by approximately 8% (67%) in both NGF or 4-AP treated cultures (FIG. 10E and FIG. 10F). NGF and 4-AP combination treatment accelerated wound closure (95-98% closure by 12 hours), compared to each agent alone. Addition of NGF neutralizing antibody attenuated this response (FIG. 10E and FIG. 10F). This means that 4-AP mediated effects on scratch closure are not 100% dependent on NGF signalling in keratinocytes.

Together these results demonstrate that 4-AP can be used for cutaneous wound healing and tissue regeneration. For example, a composition including 4-AP can be administered to a mammal (e.g., a human) to promote cellular proliferation, migration, and de-differentiation.

### Materials and Methods

### Study Design

The primary objective of this study was to investigate the possible therapeutic effect of 4-AP in enhancing skin wound healing and tissue regeneration in C57BL/6 male mice as a form of reinnervation-stimulator neurotrophins and regenerative cells medicine. The therapeutic effect of 4-AP in keratinocytes proliferation, reinneravation, neurotrophic factors expression in mice model and by in-vitro experiments was also investigated. Mice were age-matched and randomized before treatment into different groups. Data were generated by microscopic analysis of immunohistochemistry, immuno-fluorescence on fixed skin sections, and Western blotting of harvested tissue, and human skin primary cells extracts used. Sample sizes were chosen empirically to ensure adequate statistical power and were in the line with field standards for the techniques used in the study. Sample sizes for each experiment are included in the figure legends.

### Mice Skin Wound Healing Model

Male C57BL/6 (10 week) mice were purchased from the Jackson Laboratory (Bar Harbor, ME USA). Mice were anesthetized by intraperitoneal injection of ketamine (60 mg/kg) and xylazine (4 mg/kg) body weight, and the dorsal skin hair was removed using mechanical shaver and then, applied the hair removal cream. Skin was disinfected using 70% ethanol and betadine for 3 times. The dorsal skin was folded and raised cranially and caudally at midline using index fingers and thumbs, and place the animal in a lateral position and two 5-mm wounds created in their dorsal skin using sterile biopsy punch. A 5-mm-diameter silicone ring was then placed and sutured around the each wound to restrict contraction. After wound creation and sutured silicone ring, wound site was photographed, and wound surface was covered with a Tegaderm (3M) sterile transparent dressing. After surgery, mice was administered SR Buprenorphine (0.05 mg/kg) as a post-operative analgesia. The mice were randomized into the following two groups: a control group (vehicle control), which received 100 μl of saline, and a 4-AP group. In total 40 g/mouse/daily 4-AP in 90-110 μl of saline intraperitoneally (IP) was administered until day 14 post wound. Digital imaging of the wound was used was determined for each wound healing by quantification of wound area based on digital pictures taken from day 0 to day 14 (days 0, 3, 5, 7, 9, 12 and 14) post-surgery (FIG. 3A). Wound areas were measured in pixel using ImageJ-1.53e software (National Institutes of Health, USA) and normalized/corrected each wound area with reference scale and wound healing expressed as percentage with respect to day 0 wound, using following formula.

$$Wound healing ( \% ) = \frac{\begin{matrix}( Area of original wound at day 0 - \\ Area of wound at postulated day )\end{matrix}}{Area of original wound at day 0} \times 100$$

### Histomorphometry Analysis

At the day 14 wound skin sample were collected after mice were euthanized using CO₂ and skin samples were excised, either flash frozen or fixed in a buffered 4% formaldehyde solution at 4° C., overnight for samples to be embedded in paraffin and the wound skin paraffin blocks were processed into serially cut into 5 m sections on a Microtome (Leica RM2235, Germany). The wound skin sections were used for morphometric analysis and immunofluorescence staining. 5 m sectioned wound skin samples of day 14 were sections were deparaffinised using xylene and ethyl alcohol and stained with hematoxylin and eosin (H&E). To evaluate the epidermis thickness, and wound induced hair follicle formation, four randomly fields of each H&E-stained sections were imaged under a light microscope (Olympus BX53, Olympus, Tokyo, Japan) at 1000 magnification. The values of each mice were averaged, and then groups were compared. To measure the wound induced hair follicles, similar to above procedure, the number of hair follicles at dermis were counted and then average value calculated for each group. To understand the collagen formation, maturation and deposition Masson's trichome analysis were performed as per manufacture instructions (Sigma-Aldrich, Catalog No.: HT15-1KT).

### Culture Experiments

Foreskin collection and preparation—Newly born babies foreskin was collected after parental consent and were kept in eppendorff tube containing DMEM basal medium and immediately foreskin transported to cell culture laboratory at 4° C. for cells isolation. The fore skin was rinsed gently with 1×-PBS containing antibiotic. The skin was exposed the dermis and hypodermis. The whole hypodermis and blood vessels were removed. Subsequently, the skin cut into 1-2 mm pieces and the placed in DMEM medium with dispase-I at 4° C. overnight) for 12-18 hours). The dispase-I overnight treatment, the epidermis was separated from the dermis.

Keratinocytes isolation, culture conditions and characterization—The isolated epidermis was placed in a petri dish containing HEPES buffer for 10 minutes at room temperature, then treated with trypsin at 37° C. until the epidermis became loose and medium cloudy due to keratinocytes release. The cloudy medium was collected and trypsin activity neutralized using fetal bovine serum (FBS) in 1:1 ratio. The epidermis suspended keratinocytes were centrifuges at 1500 rpm for 5 minutes. The pellet resuspended in KGM-GOLD keratinocytes medium (Lonza KGM gold and supplements, Catlog No. 00192151 and 00192152). The isolated foreskin cultured on a culture dish and placed at 37° C. in 5% CO₂ incubator for 1-2 days to allow keratinocytes to adhere. Adhere keratinocytes were continued incubation in KGM-Gold medium until cells reach about 80% confluent.

Dermal Schwann cells isolation culture conditions and characterization—The separated dermis was minced into small pieces and placed in a petri dish containing collagenase in DMEM basal medium at 37° C. for 2.5 hours. The dermis was completely dissociated and the medium was cloudy. The dissociated medium was collected in tube and centrifuged at 1500 rpm for 5 minutes. The pellet was resuspended in complete DMEM medium, added on poly-L-lysine coated dish and the dish was incubated at 37° C. in 5% CO₂ incubator for overnight. Next day, adhered cells were treated with 10 μM cytosine arabinoside containing DMEM complete medium and incubated at 37° C. in 5% CO₂ incubator for 24 hours. After, the cells were cultured in Schwann cells culture medium (ScienCell Research, Catlog No. 1701) until the cells reached about 95% confluence.

Dermalfibroblasts isolation culture conditions and characterization—The separated dermis was minced into small pieces and placed in a petri dish containing collagenase containing DMEM basal medium at 37° C. for 2.5 hours. The dermis was completed dissociated and the medium was cloudy. The dissociated medium was collected in tube and centrifuged at 1500 rpm for 5 minutes. The pellet was resuspended in complete fibroblast medium (ScienCell Research, Catlog No. 2331), added on culture dish and the dish was incubated at 37° C. in 5% CO₂ incubator for overnight. Next day, adhered cells continued culture complete fibroblast medium, added on culture dish and the dish was incubated at 37° C. in 5% CO₂ incubator until cells reach about 95% confluent.

Cell Viability Assay with 4-Aminopyridine

The keratinocytes, Schwann cells and fibroblast were culture on 96-well plates, the wells were pre-coated with respective coating matrix in complete growth medium and incubated cells for 12-18 hours at 37° C. in 5% CO₂ incubator. Cells were washed with PBS and added basal Schwann cells/fibroblast/keratinocytes medium to starve, then incubated for 4 hours at 37° C. with 5% CO₂. After the incubation period, various concentrations of 4-AP (concentrations ranging from 1 to 10000 μM) in 100 μL containing respective medium's (complete keratinocytes, Schwann cells and fibroblast medium), cells were incubated either 24 hours at +37° C. and 5% CO₂ incubator. After completion of incubation time, 10 μL of the MTT labeling reagent (final concentration 0.5 mg/ml) was added to each well and incubate at +37° C. for 4 hours in a humidified atmosphere. Then, 100 μL of the solubilization solution into each well and allow the plate to stand in the incubator overnight or until the formazan crystals had dissolved. Live keratinocytes/Schwann cells/fibroblast were colored with formazan purple-blue, as the dead cells do not turn the purple-blue color. The formazan absorbance was read using wavelength of 550 nm in a plate reader. The color of optical density (OD) is directly propositional to number of live cells. The percentage of live keratinocytes/Schwann cells/fibroblast cells was determined using the following formula.

Cell viability (%)=(OD of 4−AP treated cells at particular concentration−OD of medium)/OD of control cells(No treatment)−OD of medium×100

### Cells Scratch Wound Healing Migration Assay

Keratinocytes/Schwann cells/fibroblasts (7×10⁴ cells/well keratinocytes and 3.5×10⁴ cells/well of SCs and FBs) with their respective medium were seeded on pre-coated either with collagen-I/PLL/no 96-well ImageLock microplate for 6 hours (Incucyte-sartorius plate, Cat log 4379). For the drug treatment cells were pretreated with 4-AP prior to coating for 16-18 hours. Next, the wound scratch was created using IncuCyte automated system (Essen BioScience). After scratches the cells washed with PBS and the respective cells medium was added with or without 1 mM of 4-AP. The plate was incubated in Incucyte automated imaging system, wound healing and cells migration was monitored by time-lapse photography capturing every hour from 0 to 42 hours. The relative area of wound size and cells migration at each time point was analyzed using IncuCyte™ Scratch Wound Cell Migration Software Module (Essen BioScience) and the percent of wound healing was calculated from the area measured after scratching relative to the basal area as expressed in pixels.

### Cells Immuno-Fluorescence Staining

Indirect immunofluorescence was used to identify, characterize and to analyze the proliferation after 4-AP treatment. For each cells (keratinocytes/Schwann cells/fibroblasts) treated with 4-AP and untreated cultures were processed in the same experimental session. The equal number of passage-1 cells were seeded on chamber slide. The cells were grown in their respective complete medium in presence and absence of 4-AP for 72 hours. After, the cells were fixed with 4% paraformaldehyde followed by 0.1% tritonX-100 and respective primary antibodies used, such as—cytokeratin-14, keratin-10, keratin-15, S100, p75-NTR, MPZ, vimentin and α-SMA were in 5% BSA containing PBS. Then, incubated with respective secondary antibodies and washed with PBS after incubation. The chamber glass slide mounted using Prolong Gold anti-fade mounting medium with DAPI then carefully coverslip was placed.

Co-Culture of Keratinocytes with Schwann Cells or Fibroblast on Wound Scratch Assay

To determine the influence of Schwann cells and fibroblast on keratinocytes migration and proliferation wound healing scratch assay was conducted. The co-culture experiment performed in Keratinocytes to either Schwann cells or fibroblasts in a ratio of 10:1 with same proposition of their respective medium were seeded on 96-well ImageLock microplate for 6 hours and the wells were pre-coated with collagen-I plate. For the drug treatment cells were pretreated with 4-AP prior to coating for 16-18 hours. Next, the wound scratch was created and treated cells with and without 4-AP in medium. Imaged for every hour until 24 hour and analyzed for percent of closure as described above procedure.

Effect of Combination 4-AP and NGF with without NGF-Antibody on Wound Healing

Keratinocytes (7×10⁴ cells/well) with KC medium were seeded on collagen-I pre-coated 96-well ImageLock microplate for 6 hours. For the drug treatment cells were pretreated with 4-AP prior to coating for 16-18 hours. Next, the wound scratch was created using IncuCyte automated system. After scratches the cells washed with PBS and the respective cells medium was added with individual 1 mM of 4-AP and 200 ng/ml of NGF (MyBioSource, Inc., Catlog No. MBS7114974) in keratinocytes medium and combination of 1 mM of 4-AP & 200 ng/mL NGF with or without 10 μg/ml of NGF-antibody (ThermoFisher Scientific, Catlog No. MA5-32067). The plate was incubated in Incucyte automated imaging system, wound healing and cells migration imaged for every hour until 24 hour and analyzed for percent of closure as described above procedure.

### Immunohistochemistry and Immunofluorescence Staining

Briefly, immunohistochemical and immunofluorescence analysis were performed on 5 μm thick wound healing mouse skin sections. The sections were deparaffinized and subjected to an antigen retrieval process by using sodium citrate buffer at 95° C. followed by immersed in 0.5% triton X-100. The section slides were blocked using 5% goat serum in 0.1% PBS-T for 1 hour. The following primary antibodies mouse monoclonal [LL002] anti-Cytokeratin14 (catalog No.—ab7800; IF-1:100), rat CD31 antibody (Catalog No.—553370; IF-1:100), mouse S100 antibody (Catalog No.—MA5-12969; IF-1:200), rabbit nerve growth factor receptor—p75-NTR antibody (Catalog No.—AB1554; IF and WB-1:500), Chicken neurofilament Heavy-NFH antibody (Catalog No.—NB300-217; IF-1:500), mouse alpha-smooth muscle actin —α-SMA antibody (Catalog No.—14-9760-82; IF-1:200 and WB-1:500), mouse TGF beta antibody (Catalog No.—27969; IF-1:100 and WB-1:500), rabbit Ki67 antibody (Catalog No.—9129S; IF-1:400), chicken keratin 15 antibody (Catalog No. —833901; IF-1:500), rabbit K17 antibody (gift from Pierre a. coulombe Lab; IF-1:1000), rabbit interleukin-1 beta antibody (Catalog No.—GTX74034; IF-1:100), rat F4/80 antibody (Catalog No.—MCA497GA; IF-1:100), mouse anti-SOX10 antibody (Catalog No.—sc-365692; IF-1:100 and WB-1:200), chicken anti-myelin basic protein antibody (Catalog No.—MBP; IF-1:1000), rabbit vimentin antibody (Catalog No.—10366-1-AP; IF-1:200), rabbit NGF antibody (Catalog No.—MA5-32067; IF-1:100 and WB-1:1000), mouse PGP 9.5 antibody (Catalog No.—PA5-29012; IF-1:200), chicken anti-P-zero myelin protein antibody (Catalog No.—PZO; IF-1:200), and rat substance P antibody (Catalog No. —NB100-65219; IF-1:100) were overnight incubated with 5% BSA in 0.1% PBS-T for IF stain and/or 5% skimmed milk in 0.1% TBS-T for Western blot at 4° C., then incubated with secondary antibodies for 1 hour at room temperature. The ProLong™ Gold Anti-fade Mountant with DAPI (Invitrogen, Caltlog No. P36935) used as nuclear counterstain. The immunofluorescence stained sections were imaged using ZEISS Axio Observer 7—Axiocam 506 mono—Apotome.2 microscope. The image analysis and quantification were formed either using ZEN 2.6 pro (Zeiss) imaging software or ImageJ-1.53e software (National Institutes of Health, USA).

### Protein Isolation and Western Blot

For protein isolation the harvested skin tissue was flash frozen immediately. The frozen skin tissue ground to a fine power using a liquid nitrogen mortar. The harvested cells and/or tissue powder dissolved in RIPA buffer containing Halt™ Phosphatase (Thermo Scientific, Catlog No. 78420,) and Protease Inhibitor Cocktail (Roche complete tablets mini EASYpack, Catlog No. 04694124001). Tissue and cell debris removed by centrifuged at 14000 rpm for 30 minutes at 4° C. The supernatant was collected and the total protein concentration was determined by BCA protein assay (Thermo Scientific™ Pierce™, Catlog No. 23225). The proteins (20-30 μg) of the tissue protein samples were subjected to 12% sodium dodecyl sulfate polyacrylamide gel electrophoresis (Bio-Rad mini-PROTEAN TGX Gels, Catlog No. 4561044) and transferred to polyvinylidene fluoride (PVDF) membranes. After the membranes were blocked with 5% skimmed milk in 1×TBS-T for 1 hour, they were incubated with the appropriate primary antibodies (1:200-1:1000) at 4° C. overnight, then incubated with HRP-conjugated secondary antibodies (1:3000) for 1 hour. Immunoreactivity was then detected using chemiluminescent substrate (Thermo Scientific™ SuperSignal™ West Pico PLUS, Catlog No. 34577). The intensities of the bands were quantified using Gel-imaging software. The quantified band intensity were normalized using GAPDH and expressed either normalized intensity or ratios with respect to saline treated mice.

### Statistical Analysis

Statistical analysis was performed and graphs created using Prism 7 software (GraphPad Software Inc., San Diego, CA) and data presented as means±SEM. For wound healing multiple time-point comparission study, sidak's test, and two-way analysis of variance (ANOVA) was used for analyses. Unpaired t test was performed to compare two groups, and one-way analysis of variance (ANOVA) was used for analyses. Sample sizes are indicated in the figure legends. P<0.05 was considered statistically significant for all reported analyses, as specified in the corresponding figure and figure legends.

### Example 3: 4-AP Promotes Hair Regrowth and Generation

4-AP is incorporated into a formulation designed for local administration.

To apply 4-AP to skin, 4-AP is prepared in either in isopropyl alcohol-propylene glycol-water solution or currently available balms (compatible with skin). The resulting homogenous clear solutions are passed through 2.5 micron polypropylene filters and filled in to Amber colored capacity bottles (4-AP is sensitive to light) and a total dose of about 1 mL.

For localized topical administration, the 4-AP formulated homogeneous solution is applied twice daily to skin after shaving, beginning at the center of the area. The area of application is the size of the affected area. The 4-AP formulated solution is allowed to dry for 2 to 4 hours after application. For application to the scalp, the topical application of 4-AP application continues for 21 days. For application after shaving, the topical application of 4-AP application continues for 2-4 days.

For localized injection, the 4-AP formulated homogeneous solution is injected subcutaneously in small doses under shaved skin. The 4-AP solution is delivered using a PLGA based thermogelling-delivery vehicle.

The 4-AP formulated homogeneous solution is applied locally by both topical administration and subcutaneous injection to skin grafts to induce hair grow on grafted skin.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating hair loss, wherein said method comprises (a) identifying a mammal as having hair loss, and (b) administering a composition comprising 4-AP or one or more derivatives of 4-AP to said mammal.

2. The method of claim 1, wherein said mammal is a human.

3. The method of claim 1, wherein said administering comprises a systemic administration or a local administration.

4. (canceled)

5. The method of claim 1, wherein said composition is effective to deliver about 0.05 mg/kg to about 1 mg/kg of said 4-AP or said one or more derivatives of 4-AP to said mammal.

6. The method of claim 1, wherein said mammal has a disease, disorder, or condition associated with said hair loss, and optionally wherein said disease, disorder, or condition associated with said hair loss is selected from the group consisting of traction alopecia, alopecia areata, trichotillomania, a skin graft, and a scar.

7. (canceled)

8. The method of claim 1, wherein said mammal is administered an additional medical treatment, and optionally wherein said additional medical treatment is selected from the group consisting of radiation therapy, a chemotherapy drug, a hormone therapy, Vitamin A, an acne medication, an antibiotic, an antifungal, an anticoagulant, a cholesterol-lowering drug, an immunosuppressant, an anticonvulsant, a blood pressure medication, an antidepressant, and a weight loss drug.

9. (canceled)

10. A method for increasing hair growth or increasing hair regeneration, wherein said method comprises administering a composition comprising 4-AP or one or more derivatives of 4-AP to a mammal identified as being in need of increased hair growth or in need of increased hair regeneration.

11. The method of claim 10, wherein said method is effective to increase hair growth or to increase a number of hair follicles on said mammal by at least 1.5 fold.

12. The method of claim 10, wherein said mammal is a human.

13. The method of claim 10, wherein said administering comprises a systemic administration or a local administration.

14. (canceled)

15. The method of claim 10, wherein said composition is effective to deliver about 0.05 mg/kg to about 1 mg/kg of said 4-AP or said one or more derivatives of 4-AP to said mammal.

16. The method of claim 10, wherein said mammal has a disease, disorder, or condition associated with said hair loss, and optionally wherein said disease, disorder, or condition associated with said hair loss is selected from the group consisting of traction alopecia, alopecia areata, trichotillomania, a skin graft, and a scar.

17. (canceled)

18. The method of claim 10, wherein said mammal is administered an additional medical treatment, and optionally wherein said additional medical treatment is selected from the group consisting of radiation therapy, a chemotherapy drug, a hormone therapy, Vitamin A, an acne medication, an antibiotic, an antifungal, an anticoagulant, a cholesterol-lowering drug, an immunosuppressant, an anticonvulsant, a blood pressure medication, an antidepressant, and a weight loss drug.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method for promoting proliferation or migration of a cell or promoting a hair follicle in a mammal, wherein said method comprises administering a composition comprising 4-AP or one or more derivatives of 4-AP to a mammal, optionally wherein said cell is selected from a keratinocyte, a Schwann cell, and fibroblast, and optionally wherein said cell of said mammal proliferates or migrates.

31. The method of claim 30, wherein said method comprises identifying said mammal as being in need of proliferation of said cell of said mammal prior to said administering or as being in need of migration of said cell in a lung of said mammal prior to said administering.

32. The method of claim 30, wherein said mammal is a human.

33. The method of claim 30, wherein said administering comprises a systemic administration or a local administration.

34. (canceled)

35. (canceled)

36. (canceled)

37. (canceled)

38. (canceled)

39. (canceled)

40. The method of claim 30, wherein said method comprises promoting re-epithelization of a hair follicle in a mammal, and wherein said hair follicle of said mammal undergoes re-epithelization, and optionally wherein said method comprises identifying said mammal as being in need of hair follicle re-epithelization prior to said administering.

41. (canceled)

42. (canceled)

43. (canceled)

44. (canceled)

45. The method of claim 30, wherein said method comprises promoting hair follicle formation in a mammal, and wherein a hair-follicle is formed within said mammal, and optionally wherein said method comprises identifying said mammal as being in need of hair-follicle formation prior to said administering.

46. (canceled)

47. (canceled)

48. (canceled)

49. (canceled)

50. The method of claim 30, wherein said method comprises promoting angiogenesis in a hair follicle of a mammal or promoting reinnervation of a hair follicle in a mammal, and wherein said hair follicle of said mammal undergoes angiogenesis or undergoes reinnervation, and optionally wherein said method comprises identifying said mammal as being in need of hair follicle angiogenesis prior to said administering or as being in need of hair follicle reinnervation prior to said administering.

51. (canceled)

52. (canceled)

53. (canceled)

54. (canceled)

55. (canceled)

56. (canceled)

57. (canceled)

58. (canceled)

59. (canceled)
